(19) United States
(12) Patent Application Publication
(10) Pub. No.: US 2024/0247182 A1
(43) Pub. Date: Jul. 25, 2024

(54) FRACTURING METHODS USING TAGGED POLYMERS

(71) Applicant: Kemira Oyj, Helsinki (FI)

(72) Inventors: Mehrdad HESAMPOUR, Espoo (FI); Vesa NUUTINEN, Espoo (FI); Mikko KARESOJA, Espoo (FI); Roopa TELLAKULA, Suwanee, GA (US); Rick GRIFFIN, Kennesaw, GA (US); Justin SHEPHERD, Houston, TX (US); Paula HENDERSON, Houston, TX (US); Erkki METSÄLÄ, Helsinki (FI); Viktor KUKKONEN, Espoo (FI)

(21) Appl. No.: 18/562,131

(22) PCT Filed: Jun. 1, 2022
(86) PCT No.: PCT/US2022/031809

§ 371 Date: Nov. 17, 2023

Related U.S. Application Data
(60) Provisional application No. 63/195,350, filed on Jun. 1, 2021.
(60) Provisional application No. 63/365,177, filed on May 23, 2022.

(51) Int. Cl. C09K 8/68; C09K 8/88

(52) U.S. Cl. CPC C09K 8/68; C09K 8/882; C09K 2208/28

(57) ABSTRACT

Tagged copolymers, polymer compositions including the tagged copolymers, methods of copolymerization, and methods of hydraulic fracturing. The copolymers may be tagged with a monomer as described herein. Two tagging monomers exhibiting peak fluorescence at different wavelengths may be used to permit monitoring of multiple wells.

### CROSS-REFERENCE TO RELATED APPLICATIONS

This application claims priority to U.S. Provisional Patent Application No. 63/195,350, filed Jun. 1, 2021, and U.S. Provisional Patent Application No. 63/365,177, filed May 23, 2022, which are incorporated by reference herein.

### FIELD OF THE INVENTION

This disclosure is directed to tagging agents and polymeric, detectable chemical compositions, including compositions that may be used in hydraulic fracturing and other applications.

### BACKGROUND

In the drilling, completion, and stimulation of oil and gas wells, well treatment fluids are often pumped into well bore holes under high pressure and at high flow rates causing the rock formation surrounding the well bore to fracture. As the fluid is pumped through the pipe at high flow rates there is a significant amount of frictional resistance, which can result in large energy requirements.

Friction reducing additives have been known and added to well treatment fluids to reduce pump pressure. For example, a type of well treatment commonly utilized for stimulating hydrocarbon production from a subterranean zone penetrated by a well bore is hydraulic fracturing. Hydraulic fracturing, also referred to as fracing (or fracking), is used to initiate production in low-permeability reservoirs and re-stimulate production in older producing wells. In hydraulic fracing, a fluid composition is injected into the well at pressures effective to cause fractures in the surrounding rock formation. Fracing is used both to open up fractures already present in the formation and to create new fractures.

Water soluble polymers can be used as friction reducers in well treatment fluids to alter the rheological properties of the fluid so that the turbulent flow is reduced, thereby preventing consequent energy loss in the fluid as it is pumped through the pipe. In some instances, water soluble friction reducing polymers are dispersed in water-in-oil emulsions, wherein upon addition to the aqueous treatment fluid, the emulsion inverts to release the friction reducing polymer into the fluid.

Hydraulic fracturing typically is done in stages, and, as a result, having data on oil production from different sections/zone of wells can be helpful for operators planning subsequent fracturing work.

As operators drill wells with tighter spacing and larger fracs are pumped, interfield communication becomes a greater concern. The connectivity between two or more wells can impact overall performance, and, in some instances, increase the operational cost.

Hydraulic fracturing operators often need to acquire information about connectivity of wells. One way to get this information is to monitor/detect a friction reducer on production wells in order to identify breakthrough and propagation of polymer through a reservoir.

However, polymer analysis typically requires advanced equipment, which is not usually available in fields. As decisions need to be made with haste, the use of an onsite measurement would be beneficial.

There remains a need for tagged polymers, which may be used as, or as part of, friction reducing materials, that can be detected/monitored with an instrument, preferably an onsite instrument, such as an onsite instrument capable of performing fluorescence spectroscopy, to provide for a quick measurement of tagged polymers. There also is a need for improved methods for obtaining information regarding multiple wells. For example, there remains a need for methods of assessing communication among wells in the same reservoir, which may allow operators to make any desirable adjustments to the size of a fracturing job and/or well spacing.

### BRIEF SUMMARY

Provided herein are tagged polymers, which may be used as or in a polymer composition, such as a well treatment fluid. The tagged polymers provided herein can be detected/monitored with fluorescence spectroscopy, which may be an onsite instrument. The use of an onsite instrument can allow a user to perform a quick, onsite measurement of tagged polymer. The tagged polymers herein also may permit “multi-tagging” of at least two different polymers (i.e., each polymer having a different tag), which may be used to monitor/detect the tagged polymers in separate wells. This may permit a degree of communication among two or more wells to be assessed. Additionally or alternatively, the compositions herein may be used to assess stages in a well. The tagged polymers provided herein may permit the quick, easy, and/or accurate measurement of polymer concentration, including the concentration of tagged polymers having a variety of molecular weights. The compounds and polymer compositions provided herein may be suitable for a number of applications, including, but not limited to, hydraulic fracturing. The compounds provided herein and/or derivatives thereof may be used as biomarkers.

In one aspect, polymer compositions are provided. In some embodiments, the polymer compositions include a copolymer. The copolymer may include (i) a first monomer comprising (a) a compound of formula (I), formula (I′), or formula (I″), or (b) a compound or an isomer of formula (II), wherein the first monomer is a tagging monomer, and (ii) at least one second monomer comprising at least one polymerizable double bond or at least one polymerizable triple bond;

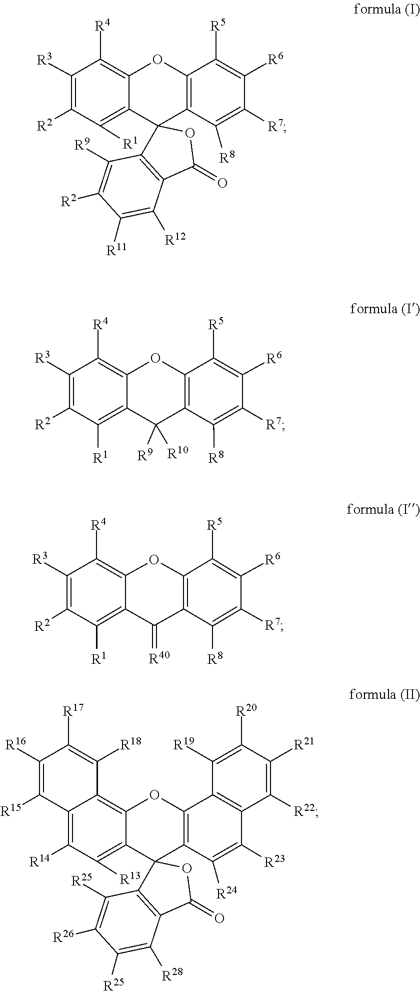

wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R₂₂, R¹⁹, R²⁰, R²¹, R²², R²³, R²⁴, R²⁵, R²⁶, R²⁷, and R²⁸ are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl, wherein R⁴⁰ is selected from the group consisting of C₁-C₆ alkoxy, C₂-C₆ alkenoxy, C₂-C₆ alkynoxy, —N(R′), —N(R′)(R″), C₂-C₆ alkyl, C₂-C₆ alkenyl, C₂-C₆ alkynyl, and C₄-C₁₄ aryl, wherein R′ and R″ are independently selected from the group consisting of hydrogen and C₁-C₆ alkyl, and wherein the isomers of Formula (II) comprise a compound of Formula (IIi), a compound of Formula (IIii), a compound of Formula (Iliii), or a combination thereof—

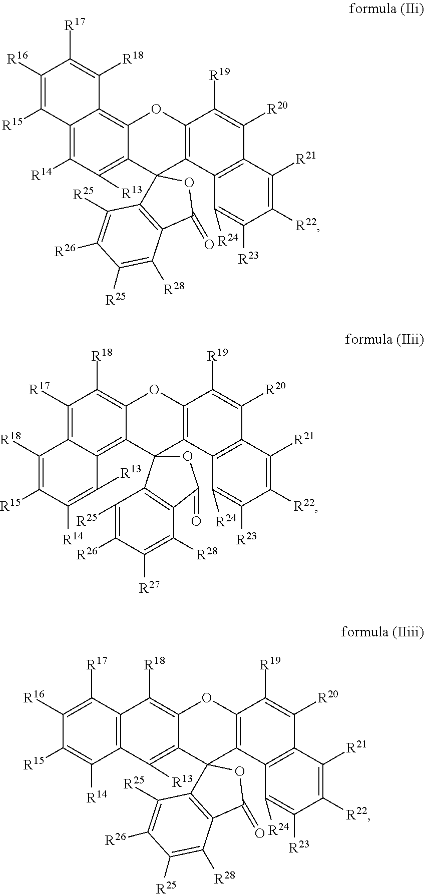

In another aspect, well treatment fluids are provided. The well treatment fluids may include a polymer composition described herein; for example, a well treatment fluid may include a copolymer as described herein, a fluid as described herein, and optionally one or more additional components described herein, such as a surfactant. A well treatment fluid described herein may be formed by providing a water-in-oil emulsion produced by a copolymerization method described herein, and disposing the water-in-oil emulsion in a aqueous fluid to form the well treatment fluid.

In a further aspect, methods of hydraulic fracturing are provided. In some embodiments, the methods include providing a well treatment fluid described herein; disposing the well treatment fluid in a well of a reservoir; and measuring with an analytical technique an amount of the first monomer in the well treatment fluid after the disposing of the well treatment fluid in the well to (i) identify one or more producing stages, (ii) determine an optimum concentration of the copolymer in the well treatment fluid, or (iii) determine whether the well treatment fluid is recyclable.

In some embodiments, the methods of hydraulic fracturing include providing a first well treatment fluid described herein; providing a second well treatment fluid described herein, wherein (A) a peak fluorescence emission of the first monomer of the first well treatment fluid and (B) a peak fluorescence emission of the first monomer of the second well treatment fluid occur at different wavelengths; disposing the first well treatment fluid in a first well of a reservoir; disposing the second well treatment fluid in a second well of the reservoir; and measuring with an analytical technique (i) an amount of the first monomer of the first well treatment fluid and (ii) an amount of the first monomer of the second well treatment fluid after the disposing of the first and the second well treatment fluids in the first and the second wells, respectively, to determine (a) a degree of connectivity between the first well and the second well of the reservoir, or (b) one or more producing stages of the first well or the second well.

In a still further aspect, methods of polymerization are provided. In some embodiments, the methods include providing an aqueous phase having a first pH, the aqueous phase comprising water and at least one second monomer, as described herein; optionally adjusting the first pH to a second pH; disposing a first monomer as described herein in the aqueous phase; providing an oil phase that includes an oil and at least one emulsifying surfactant; combining the aqueous phase and the oil phase to form an emulsion; optionally deoxygenizing the emulsion; disposing an initiator in the emulsion; and contacting the emulsion with an initiator, such as a redox couple, to copolymerize the first monomer and the at least one second monomer to form a copolymer as described herein. The emulsion in which the copolymerization occurs may be a water-in-oil emulsion that can be used to produce a well treatment fluid.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1A depicts a plot comparing the fluorescence yield of an embodiment of a copolymer uncleaned or cleaned via size-exclusion chromatography.

FIG. 1B depicts a plot comparing the fluorescence yield of an embodiment of a copolymer uncleaned or cleaned via size-exclusion chromatography.

FIG. 2 depicts a plot of intensity v. emission for an embodiment of a copolymer described herein.

FIG. 3 depicts a plot of intensity v. emission for an embodiment of an untagged polymer.

FIG. 4 depicts a plot of intensity v. emission for an embodiment of a copolymer described herein.

FIG. 5 depicts a correlation between intensity and concentration of an embodiment of a copolymer described herein.

FIG. 6A and FIG. 6B depict plots of friction reduction performance of untagged polymer and an embodiment of a copolymer described herein in tap water (FIG. 6A) and 30K brine (FIG. 6B).

FIG. 7A and FIG. 7B depict a plots of friction reduction performance of untagged polymer and an embodiment of a copolymer described herein in tap water (FIG. 7A) and 30K brine (FIG. 7B).

### DETAILED DESCRIPTION

Provided herein are compounds that may be used as tagging agents, polymers that include the compounds, polymer compositions, such as well treatment fluids, that include the polymers, methods for forming the compounds and polymers, and methods for monitoring a concentration of the compounds, polymers, or polymer compositions.

### Compounds

Compounds are provided herein, which may be used as fluorescent tagging monomers in polymers, including those disclosed herein. The compounds provided herein may be present as a monomer (e.g. a comonomer and/or end group) of the polymers described herein. As used herein, the phrases “tagging agent”, “tagging monomer”, and the like refer to a compound and/or monomer that is detectable at a desirable concentration (e.g., a relatively low concentration) using an analytical technique, such as fluorescence spectroscopy.

The tagging monomers provided herein, in some embodiments, exhibit a fluorescence emission maximum at about 300 nm to about 800 nm, about 350 nm to about 750 nm, about nm to about 700 nm, about 410 nm to about 680 nm, about 410 nm to about 600 nm, about 410 nm to about 590 nm, about 410 nm to about 520 nm, about 410 nm to about 500 nm, about 440 nm to about 450 nm, about 500 nm to about 520 nm, about 550 nm to about nm, about 640 nm to about 680 nm, or about 570 nm to about 600 nm, thereby providing polymer compositions or other products with a feature that may permit an amount (e.g., a concentration) of a polymer composition that includes a tagging monomers to be monitored. In some embodiments, the excitation and emission wavelengths are determined, and, therefore, may be adjusted, by selecting a particular aryl alcohol/amino aryl alcohol. The fluorescence emission may be affected by pH; for example, the keto-enol tautomers described herein may exhibit different fluorescence emissions.

The compounds, including tagging monomers, provided herein include compounds or isomers of formula (I), formula (I′), formula (I″), or formula (II). The phrases “compound of formula (I)”, “compound of formula (I′)”, “compound of formula (I”)″, “compound of formula (II)”, the term “compound” when it refers to formula (I) or (II), the term “isomer” when it refers to formula (II), and the like, as used herein, refer to and include compounds according to or isomers of, respectively, the structures of each formula, salts thereof, hydrates thereof, salt hydrates thereof, stereoisomers thereof, dehydrates thereof, and derivatives thereof. Therefore, the formulas and structures provided herein encompass and read on the formulas and structures as drawn or isomers of the formulas and structures as drawn, as well as salts, hydrates, salt hydrates, stereoisomers, dehydrates, tautomers, or derivatives of each formula and structure or isomer thereof. The “derivatives” of each formula and structure include, but are not limited, to polymers (e.g., oligomers, copolymers, etc.) formed of the compounds. The tautomers may include keto-enol tautomers.

The compounds provided herein include compounds or isomers of formula (I) or formula (II), which, as explained herein, include salts, hydrates, salt hydrates, stereoisomers, dehydrates, tautomers, and derivatives of the compounds or isomers of formula (I) and formula (II):

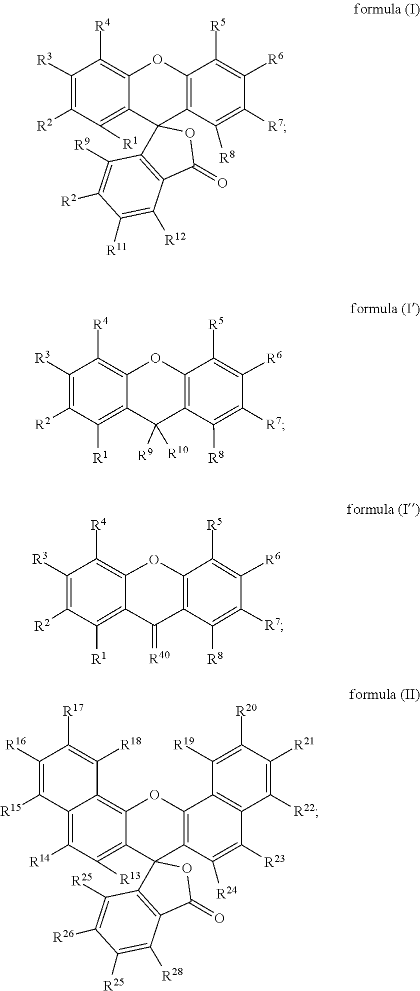

wherein R¹, R², R³, R⁴, R⁵, R⁶, R⁷, R⁸, R⁹, R¹⁰, R, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl, wherein R⁴⁰ is selected from the group consisting of C₁-C₆ alkoxy, C₂-C₆ alkenoxy, C₂-C₆ alkynoxy, —N(R′), —N(R′)(R″), C₂-C₆, alkyl, C₂-C₆ alkenyl, C₂-C₆ alkynyl, and C₄-C₁₄ aryl, wherein R′ and R″ are independently selected from the group consisting of hydrogen and C₁-C₆ alkyl, and wherein the isomers of formula (II) comprise a compound of formula (IIi), a compound of formula (IIii), a compound of formula (Iliii), or a combination thereof—

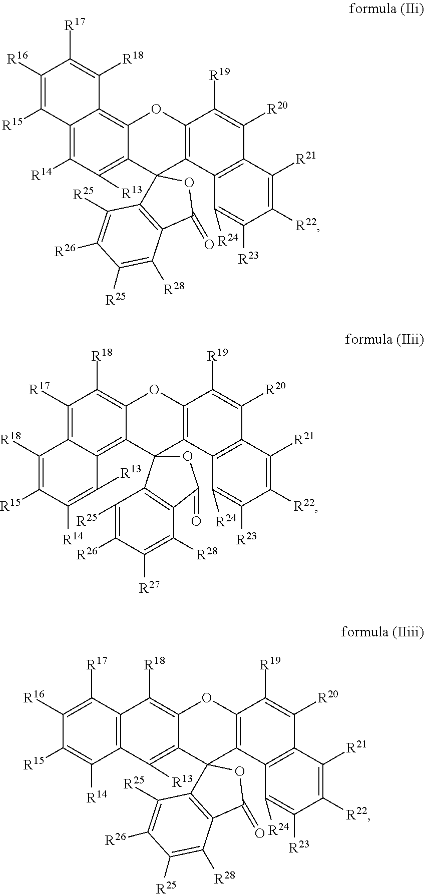

The compounds provided herein may include “R” groups (e.g., R¹, R², etc.) selected from C₁-C₆ alkoxy, C₂-C₆ alkenoxy, C₂-C₆ alkynoxy, C₁-C₆ alkyl, C₂-C₆ alkenyl, C₂-C₆ alkynyl, C₄-C₁₄ aryl, C₁-C₆ alkenyl, and the like.

Each C₁-C₆ alkoxy, C₂-C₆ alkenoxy, C₂-C₆ alkynoxy, C₁-C₆ alkyl, C₂-C₆ alkenyl, C₂-C₆ alkynyl, C₄-C₁₄ aryl, C₁-C₆ alkenyl, and the like disclosed herein, includes all substituted, unsubstituted, branched, and linear analogs or derivatives thereof, in each instance having the indicated number of carbon atoms. Examples of alkyl groups include, but are not limited to, methyl, ethyl, propyl, isopropyl, n-butyl, t-butyl, isobutyl, pentyl, hexyl, isohexyl, cyclopropyl, cyclobutyl, cyclopentyl, and cyclohexyl. Additional examples of alkyl moieties have linear, branched and/or cyclic portions. Representative alkenyl moieties include, but are not limited to, vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and 1-hexenyl. Representative alkynyl moieties include, but are not limited to, acetylenyl, propynyl, 1-butynyl, 2-butynyl, 1-pentynyl, 2-pentynyl, 3-methyl-1-butynyl, 4-pentynyl, 1-hexynyl, 2-hexynyl, and 5-hexynyl. Examples of alkoxy, alkenoxy, and alkyoxy compounds include any of the foregoing alkyl groups, alkenyl groups, or aklynyl groups that are covalently bonded to an oxygen atom. Examples of aryl moieties include, but are not limited to, anthracenyl, azulenyl, biphenyl, fluorenyl, indan, indenyl, naphthyl, phenyl, 1,2,3,4-tetrahydro-naphthalene, and the like, including substituted derivatives thereof, in each instance having from 4 to about 14 carbons. Substituted derivatives of aromatic compounds include, but are not limited to, tolyl, xylyl, mesityl, and the like, including any heteroatom substituted derivative thereof.

Each C₄-C₁₄ aryl group of the compounds provided herein may independently include (i) a single “R” substituent (for example, one of R¹, R², R³, R⁴, R⁷, or R⁸), or (ii) at least two “R” substituents on adjacent carbon atoms (for example, R¹ and R², wherein R¹ and R² are covalently bonded to each other; R² and R³, wherein R² and R³ are covalently bonded to each other; R³ and R⁴, wherein R³ and R⁴ are covalently bonded to each other; etc.). Therefore, for example, in formula (I), an unsubstituted C₆ aryl group (i.e., a phenyl) may be selected for each of R² and R³ (Structure (a)), or an unsubstituted C₄ aryl group may be selected jointly for R² and R³, thereby resulting in a 6-membered aryl ring that includes the carbon atom to which R² is covalently bonded, the carbon atom to which R³ is covalently bonded, R², and R³, wherein R² and R³ are covalently bonded to each other (Structure (b)):

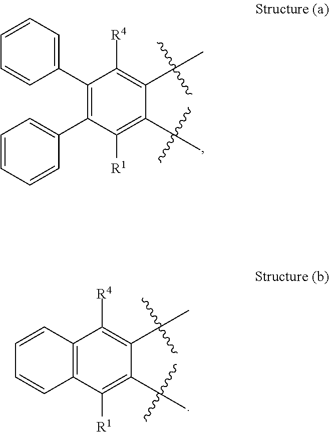

Each C₁-C₆ alkoxy, C₂-C₆ alkenoxy, C₁-C₆ alkyl, C₂-C₆ alkenyl, and/or C₁-C₆ alkenyl of the compounds provided herein may independently include (i) a single “R” substituent (for example, one of R′, R″, R¹, R², R³, R⁴, R⁷, or R⁸), or (ii) at least two “R” substituents on adjacent carbon atoms (for example, R¹ and R², wherein R¹ and R² are covalently bonded to each other; R² and R³, wherein R² and R³ are covalently bonded to each other; R³ and R⁴, wherein R³ and R⁴ are covalently bonded to each other; R′ and R², wherein R′ and R² are covalently bonded to each other, etc.). Therefore, for example, in formula (I), an unsubstituted C₆ alkyl group (i.e., a hexyl) may be selected for each of R² and R³ (Structure (a)), or an unsubstituted C₄ alkyl group may be selected jointly for R² and R³, thereby resulting in a 6-membered ring that includes the carbon atom to which R² is covalently bonded, the carbon atom to which R³ is covalently bonded, R², and R³, wherein R² and R³ are covalently bonded to each other (Structure (b)):

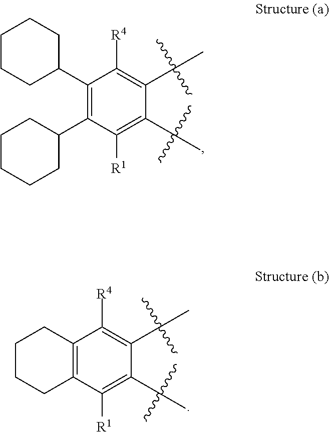

In some embodiments, any two adjacent “R” groups are not bonded to each other. For example, substituents R⁹ and R¹⁰ of formula (I′) may not be bonded to each other.

When used herein with regard to the selection of a substituent, the term “independently” indicates that (i) a substituent at a particular location may be the same or different for each molecule or monomer of a formula (e.g., (i) a compound of formula (I) may include two molecules of formula (I), with each molecule having the same or a different hydrocarbyl selected for R¹; or (ii) a polymer including formula (I) may include two monomers of formula (I), with each monomer having the same or a different hydrocarbyl selected for R¹), and/or (ii) two differently labeled substituents selected from the same pool of substituents may be the same or different (e.g., R¹ and R² of a monomer may both be selected from “a C₁-C₆ alkyl”, and the C₁-C₆ alkyls selected for R¹ and R² may be the same or different).

Unless otherwise indicated, the term “substituted,” when used to describe a chemical structure or moiety, refers to a derivative of that structure or moiety wherein (i) a multi-valent non-carbon atom (e.g., oxygen, nitrogen, sulfur, phosphorus, etc.) is bonded to one or more carbon atoms of the chemical structure or moiety (e.g., a “substituted” C₄ alkyl may include, but is not limited to, diethyl ether moiety, a methyl propionate moiety, an N,N-dimethylacetamide moiety, a butoxy moiety, etc.) or (ii) one or more of its hydrogen atoms (e.g., chlorobenzene may be characterized generally as an aryl C₆ aryl “substituted” with a chlorine atom) is substituted with a chemical moiety or functional group such as alcohol, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl— or —alkylNHC(O)alkyl), tertiary amine (such as alkylamino, arylamino, arylalkylamino), aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carboxyl, carboxylic acid, cyano, ester, ether (e.g., methoxy, ethoxy), halo, haloalkyl (e.g., —CCl₃, —CF₃, —C(CF₃)₃), heteroalkyl, isocyanate, isothiocyanate, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) or urea (—NHCONH-alkyl-). When an “R” group (e.g., R¹) is substituted, the carbon atoms in the substituents are included in the total count of carbon atoms in the “R” group. For example, if R¹ is selected from a C₁-C₆ alkyl, and the C₁-C₆ alkyl is a propyl group substituted with a dimethylamine substituent, then R¹ is considered, in this example, to be a C₅ alkyl because there are 3 carbon atoms in the propyl group, and 2 carbon atoms in the dimethylamine substituent.

When a compound or isomer of formula (I), formula (I′), formula (I″), or formula (II) includes a stereocenter, the compounds or isomers of formula (I), formula (I′), formula (I″), or formula (II) include both of the (R) and (S) enantiomers. For example, Formula (Iliii), as depicted herein, includes both of the following stereoisomers:

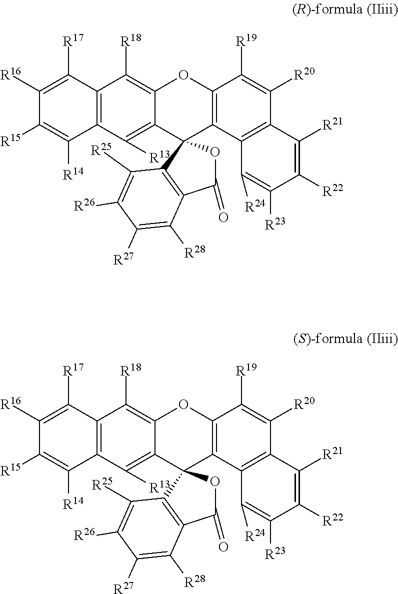

In some embodiments, R¹, R², R⁴, R, R⁷, R, R⁹, R¹⁰, R¹¹, and R¹² are hydrogen, and the compound is of formula (Ia):

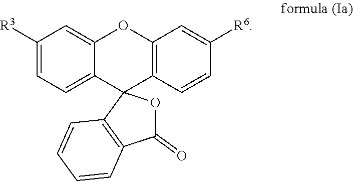

In some embodiments, the compound is of formula (I), formula (I′), formula (I″), or formula (Ia), wherein R³ and R⁶ are hydroxyl.

In some embodiments, the compound is of formula (I), formula (I′), or formula (Ia), wherein R³ and R⁶ are —N(R′)(R″). R′ and R″ may be independently selected from hydrogen and an unsubstituted C₁-C₆ alkyl, such as an unsubstituted C₂ alkyl.

In some embodiments, the compound is of formula (I′), wherein (i) R⁹ is hydrogen and R¹⁰ is methyl, (ii) at least one of R⁹ and R¹⁰ is a lactone ring, such as a C₅+ lactone ring, or (iii) R⁹ is hydroxyl and R¹⁰ is hydrogen.

In some embodiments R¹³, R¹⁴, R¹⁵, R¹⁷, R¹⁸, R¹⁹, R²⁰, R²², R²³, R²⁴, R²⁵, R²⁶, R²⁷, and R²⁸ are hydrogen, and the compound is of formula (IIa) or an isomer thereof, as described herein:

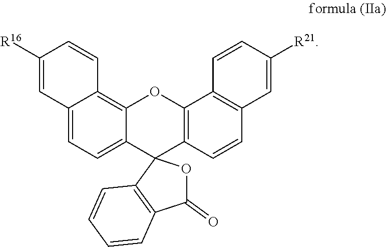

In some embodiments, R¹⁶ and R²¹ are hydroxyl in the compound or isomer of formula (II) or formula (IIa). In some embodiments, R¹⁶ and R²¹ are —N(R′)(R″) in the compound or isomer of formula (II) or formula (IIa). R′ and R″ may be an unsubstituted C₁-C₆ alkyl, such as an unsubstituted C₂ alkyl.

### Polymer Compositions

Polymer compositions are provided herein. The polymer compositions may include a copolymer, which includes a first monomer (e.g. a comonomer and/or end group) that is a tagging monomer, and at least one second monomer.

In some embodiments, the first monomer is selected from the group consisting of (a) a compound of formula (I), formula (I′), or formula (I″), and (b) a compound or isomer of formula (II), which, again, includes salts, hydrates, salt hydrates, stereoisomers, dehydrates, tautomers, or derivatives of the compounds or isomers of formulas (I) and (II). In some embodiments, the at least one second monomer includes at least one polymerizable double bond or at least one polymerizable triple bond.

In some embodiments, the copolymers are obtainable by free radical polymerization of two or more types of monomer (including 3, 4, or more different monomers) without restriction on the number of monomer units that are incorporated into the product, provided that at least one of the monomers is a first monomer (i.e., a tagging monomer) and at least one of the monomers is a second monomer, as described herein. In some embodiments, the copolymers include two or more second monomers and one or more first monomers or molecules (i.e., tagging units) as described herein.

As used herein, the terms “polymer,” “polymers,” “polymeric,” and the like are used in their ordinary sense as understood by one ordinarily skilled in the art, and thus may be used herein to refer to or describe a large molecule (or group of such molecules) that contains recurring units (i.e., monomers), including, but not limited to, oligomers, comb polymers, branched polymers, linear polymers, crosslinked polymers, star polymers, etc. Polymers may be formed in various ways, including by polymerizing monomers and/or by chemically modifying one or more recurring units of a precursor polymer. A polymer may be a “copolymer” that includes two or more different recurring units (i.e., monomers) formed by, e.g., copolymerizing two or more different monomers (e.g., 2, 3, 4, 5, 6 or more monomers), and/or by chemically modifying one or more recurring units of a precursor polymer.

The polymers, including copolymers, provided herein are defined in terms of the monomer(s) that form the structures of the polymers. Although, in the interest of clarity, monomers are depicted in isolated, unpolymerized form herein, a person ordinarily skilled in the art will understand the structural differences between the monomers in unpolymerized and polymerized forms. For example, a person ordinarily skilled in the art will understand that an embodiment of a polymerized monomer of formula (Ia) may have the following structure or a similar structure when present as an end group in the polymers described herein:

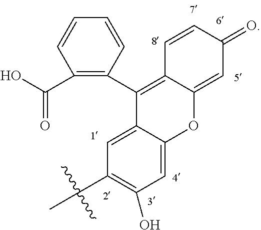 [SMILES: *c1cc2c(-c3ccccc3C(=O)O)c3ccc(=O)cc-3oc2cc1O]

Any atom of a “first monomer” may bond to another monomer, but it is believed that carbon atoms at the 2′, 4′, 5′, and 7′ positions (as depicted, for example, in the foregoing structure) have the greatest potential to react, especially with a macroradical.

The phrases “friction reducer”, “friction reduction”, “viscosifier,” “viscosifying agent,” and the like will be understood by those skilled in the art to have a broad and customary meaning that includes using the polymer compositions herein to alter one or more rheological properties of a fluid so that turbulent flow is reduced, thereby preventing or reducing consequent energy loss in the fluid as it is pumped through a pipe during a process, such as hydraulic fracturing. In some embodiments, the polymer compositions described herein, are solutions. In some embodiments, the polymer compositions described herein are dispersions (e.g., a liquid phase in which one or more at least partially undissolved components, such as a polymer, are dispersed). The polymer compositions described herein may include friction reducing polymer compositions, viscosifying polymer compositions, etc.

In some embodiments, the polymer composition is an emulsion polyacrylamide (EPAM) polymer composition.

i. First Monomer

The first monomer of the polymer compositions may include (a) a compound of formula (I), formula (I′), or formula (I″), or (b) a compound or isomer of formula (II), which, again, may include salts, hydrates, salt hydrates, stereoisomers, dehydrates, tautomers, or derivatives of the compounds or isomers of formulas (I), (II), or a combination thereof. Therefore, like the compounds described herein, the “first monomer” may be present as a comonomer and/or end group. Therefore, the first monomer, for example, may include a salt or salt hydrate of a compound or isomer of formula (I), formula (I′), formula (I″), or formula (II), such as a hydrochloride, dihydrochloride, sulfate, bisulfate, or gluconate salt, or hydrate thereof. As a further example, the first monomer may include a derivative of a compound or isomer of formula (I), (I′), or (II), such as a derivative formed from the addition of acid and heat to the compound or isomer of formula (I), (I′), or (II).

ii. Second Monomer

The at least one second monomer of the polymer compositions provided herein may include any monomer that includes a polymerizable moiety, such as a double bond or a triple bond.

In some embodiments, the at least one second monomer includes acrylamide, an acrylate (e.g., sodium acrylate), or a combination thereof. The at least one second monomer, such as an acrylate, may include any suitable countercation (e.g., Na⁺). In some embodiments, the at least one second monomer is selected from the group consisting of allylsulfonate salts, for example sodium allylsulfonate; acrylic acid; vinyl sulfonic acid; vinyl sulfonate salts; vinyl phosphoric acid; vinyl phosphonate salts; vinylidene diphosphonic acid or salts thereof; methacrylic acid; vinyl acetate; vinyl alcohol; vinyl chloride; unsaturated mono- or di-carboxylic acids or anhydrides, such as maleic anhydride, maleic acid, fumaric acid, itaconic acid, aconitic acid, mesaconic acid, citraconic acid, crotonic acid, isocrontonic acid, angelic acid, and tiglic acid; vinyl chloride; styrene-p-sulfonic acid, or styrene sulfonates salts; acrylamido-2-methylpropanesulfonic acid (ATBS); hydroxyphosphonoacetic acid (HPA); hypophosphorus acids; acrylamides; propargyl alcohol having formula HC≡C—CH₂—OH; butyr-1,4-diol, and mixtures thereof.

The polymer compositions provided herein generally may include any amount of at least one first monomer and any amount of at least one second monomer. In some embodiments, the first monomer is present in the copolymer at an amount of about 0.01% to about 10%, by weight, based on the weight of the copolymer. In some embodiments, the first monomer is present in the copolymer at an amount of about 0.005% to about 5%, by weight, based on the weight of the copolymer. In some embodiments, the first monomer is present in the copolymer at an amount of about 0.01% to about 2%, by weight, based on the weight of the copolymer. In some embodiments, the first monomer is present in the copolymer at an amount of about 0.01% to about 1.5%, by weight, based on the weight of the copolymer. In some embodiments, the first monomer is present in the copolymer at an amount of about 0.01% to about 1%, by weight, based on the weight of the copolymer. In some embodiments, the first monomer is present in the copolymer at an amount of about 0.01% to about 0.75%, by weight, based on the weight of the copolymer. In some embodiments, the first monomer is present in the copolymer at an amount of about 0.01% to about 0.5%, by weight, based on the weight of the copolymer. In some embodiments, the first monomer is present in the copolymer at an amount of about 0.01% to about 30%, about 0.01% to about 20%, about 0.01% to about 15%, about 0.01% to about 10%; about 0.01% to about 8%; about 0.01% to about 7%; about 0.01% to about 5%; about 0.01% to about 3%, or about 0.01% to about 2%, by weight, based on weight of the copolymer.

In some embodiments, the copolymer of a polymer composition has a weight average molecular weight (M_{W}) of at least 100,000 Daltons, at least 250,000 Daltons, at least 500,000 Daltons, at least 750,000 Daltons, at least 1,000,000 Daltons, at least 2,500,000 Daltons, at least 5,000,000 Daltons, at least 7,500,000 Daltons, or at least 10,000,000 Daltons. In some embodiments, the copolymer of a polymer composition has a weight average molecular weight (M_{w}) of about 100,000 Daltons to about 10,000,000 Daltons, about 500,000 Daltons to about 10,000,000 Daltons, or about 1,000,000 Daltons to about 10,000,000 Daltons.

In some embodiments, the polymer compositions may include one or more monomers, groups, or units, as necessary or desired, in addition to the first monomer and at least one second monomer. For example, the polymers may include one or more other groups resulting from a polymerization initiator, end-capping groups, or a combination thereof. In some embodiments, the end capping groups are derived from initiator compounds used in the polymerization of monomers.

The thermal stability of the polymer compositions may be evaluated by heating the polymer in a liquid, for example water or brine, to a temperature, for example, of about 80° C., about 90° C., about 100° C., about 110° C., about 120° C., or about 130° C., and keeping polymer composition in the liquid at that temperature for a period of time, for example, about one week.

In some embodiments, the polymer compositions, including the copolymers provided herein, have a thermal stability such that when a polymer composition is kept at a temperature of about 80° C. in water or brine for about one week, there is less than about a 15%, about a 10%, about a 5%, about a 4%, or about a 3% decrease in emission intensity. In some embodiments, the polymer compositions, including the copolymers provided herein, have a thermal stability such that when a polymer composition is kept at a temperature of about 130° C. in water or brine for about one week, there is less than about a 15%, about a 10%, about a 5%, about a 4%, or about a 3% decrease in emission intensity. In some embodiments, the water is at a pH of about 7 to about 8. In some embodiments, the brine is natural brine or synthetic brine. In some embodiments, the polymer compositions have a thermal stability such that when a polymer composition is kept at a temperature of about 80° C. in water for about one week, there is less than about a 10%, about a 5%, about a 4%, or about a 3% decrease in emission intensity. In some embodiments, the polymer compositions have a thermal stability such that when a polymer composition is kept at a temperature of about 130° C. in water for about one week, there is less than about a 15%, about a 10% or about a 5% decrease in emission intensity. In some embodiments, the polymer compositions have a thermal stability such that when a polymer composition is kept at a temperature of about 130° C. in water at about pH 8 for about one week, there is less than about a 15%, about a 13%, or about a 10% decrease in emission intensity. In some embodiments, the polymer compositions have a thermal stability such that when a polymer composition is kept at a temperature of about 130° C. in brine for about one week, there is less than about a 20%, about a 15% or about a 10% decrease in emission intensity.

The polymer composition may optionally include one or more additional ingredients, as necessary or desired, such as those described herein, which include water, salts, oils, surfactants, pH adjusting agents (such as acids, bases and buffers), colorants, flow modifiers, other water treatment agents, etc. In some embodiments, the polymer composition consists essentially of a copolymer that includes a first monomer and at least one second monomer, as described herein. When the polymer composition consists essentially of a copolymer that includes a first monomer and at least one second monomer, the polymer composition may include one or more of the foregoing “additional ingredients” and the following “[e]xemplary fluids”, because the “additional ingredients” and “[e]xemplary fluids” are non-limiting examples of components that do not materially affect the basic and novel characteristic(s) of the polymer compositions.

In some embodiments, the polymer compositions include (i) a copolymer of a first monomer and at least one second monomer, and (ii) a fluid. Exemplary fluids include those that may be in or intended for industrial water systems or process systems, such as boilers, cooling systems, cooling towers, desalination plants, geothermal power production, irrigation systems, mineral processing systems (such as mineral ore extraction systems), paper pulping or manufacturing systems, membrane systems, etc. Other exemplary fluids include fluids for use in the oil industry, such as those for use in the treatment of water injection systems, subsea flow lines, topside production equipment and “down-hole” to control scaling in and around the production well-bore. In some embodiments, the fluid includes tailings, such as oil and/or sand tailings. The polymer compositions may be used for polymer flooding. For example, the polymer compositions may be used as a friction reducer, a viscosifying agent, etc.

In some embodiments, the polymer compositions include an aqueous composition or a water-based fluid, for example a seawater-based fluid. Other fluids, however, are envisioned.

In some embodiments, the polymer compositions include a glycol or glycol ether based solvent.

In some embodiments, the polymer compositions include a copolymer of a first monomer and at least one second monomer, as described herein, and, optionally, one or more additional polymers. The one or more additional polymers may include a tagging agent, and the fluorescence emission of the tagging agent may differ from the fluorescence emission of the first monomer of the copolymer.

In some embodiments, the polymer composition includes one or more copolymers, as described herein, in combination with one or more additional ingredients, such as anionic surfactants (e.g. C₁₀-20 alkyl benzene sulfonates, C₁₀-20 olefin sulfonates, C₁₀-20 alkyl sulfates, C₁₀-20 alkyl 1 to 25 mole ether sulfates, C₁₀-20 paraffin sulfonates, C₁₀-20 soaps, C₁₀-20 alkyl phenol sulfates, sulfosuccinates, sulfosuccinamates, lignin sulfonates, fatty ester sulfonates, C₁₀-20 alkyl phenyl ether sulfates, C₁₀-20 alkyl ethanolamide sulfates, C₁₀-20 alpha sulfo fatty acid salts, C₁₀-20 acyl sarcosinates, isethionates, C₁₀-20 acyl taurides, C₁₀-20 alkyl hydrogen phosphates), non-ionic surfactants (e.g. ethoxylated and/or propoxylated C₁₀-20 alcohols, ethoxylated and/or propoxylated C₁₀-20 carboxylic acids, alkanolamides, amine oxides, and/or C₁₀-20 acyl sorbitan and/or glyceryl ethoxylates), amphoteric surfactants (e.g. betaines, sulfobetaines, and/or quaterised imidazolines), and/or cationic surfactants (e.g. benzalkonium salts, C₁₀-20 alkyl trimethyl ammonium salts, and/or C₁₀-20 alkyl trimethyl); sequestrants; chelating agents; corrosion inhibitors (e.g., imidazoline and quaterantry ammonium salts); and/or other threshold agents (e.g. polymers such as aminometholine phosphonate polymers, polyacrylic acid, or non polymeric agents such as sodium tripolyphosphate, sodium ethylenediamine tetracetate, sodium nitrilo triacetate, tetra potassium pyrophosphate, acetodiphosphonic acid and its salts, ammonium trismethylene phosphonic acid and its salts, ethylenediamine tetrakis (methylene phosphonic) acid and its salts, diethylenetriamine pentakis (methylene phosphonic) acid and its salts); tolyltriazole and mixtures of nitrate, benzqate, HHP and/or PTCB); hydrate inhibitors (e.g., methanol); cinetic inhibitors such as anti-agglomeration agents; biocides (e.g. tetrakis (hydroxymethyl) phosphonium salts, formaldehyde, glutaraldehyde, DENPA, bromopol isothiazoronal); oxidising biocides and/or bleaches (e.g. chlorine, chlorine dioxide, hydrogen peroxide, sodium perborate); foam controlling agents, such as silicone antifoams; oxygen scavengers such as hydrazines and/or hydroxylamines; pH controlling and/or buffering agents, such as amines, borates, citrates and/or acetates; chromium salts; zinc salts; asphaltene inhibitors; wax inhibitors; demulsifiers; and/or other water treatment agents such as polymeric dispersants and coagulants including polymaleic, polyacrylic and polyvinylsulfonic acids and their salts, starches and/or carboxy methyl cellulose, and/or molybdates.

In some embodiments, the polymer composition includes two or more copolymers. When two or more copolymers are present, each copolymer may include a different first monomer, and each of the different first monomers or molecules may exhibit a different fluorescence emission. For example, a polymer composition may include (i) a first copolymer including a first monomer, which has a fluorescence emission maximum of about 500 to about 520 nm, (ii) a second copolymer including a first monomer, which has a fluorescence emission maximum of about 550 nm to about 590 nm, (iii) a third copolymer including a first monomer, which has a fluorescence emission maximum of about 640 nm to about 680 nm, or (iv) a combination thereof. Such a polymer composition also may include an additional copolymer including a first monomer, which has a fluorescence emission maximum of about 570 nm to about 600 nm. The differences in fluorescence emission maximum may permit the methods described herein to be used to determine an amount of each copolymer present in a fluid or system, the differences between the amounts of each copolymer in a fluid or system, or a combination thereof.

In some embodiments, the polymer compositions include about 5% to about 95%, by weight, of a copolymer of a first monomer and at least one second monomer, as described herein, and about 5% to about 90%, by weight, of one or more of any of the additional ingredients described herein, based on the total weight of a polymer composition.

A copolymer of at least one first monomer and at least one second monomer may be combined with water using any suitable method. For example, a copolymer may be dissolved, suspended, dispersed, or emulsified in water. The amount of water in an aqueous polymer composition may vary, as necessary or desired. For example, an aqueous polymer composition may include about 20% to about 80%, by weight, of a copolymer of a first monomer and a second monomer, as described herein, based on the total weight of the aqueous polymer composition.

In some embodiments, the pH of a polymer composition may be such that the acidic functionalities of a copolymer, as described herein, are neutralized. For example, the composition may be neutralized by adjusting the pH of the composition to a pH in a range of about 2 to about 13. In some embodiments, the copolymer is an anionic copolymer. In some embodiments, the copolymer is a neutral copolymer. In some embodiments, the copolymer is a cationic copolymer.

The polymer compositions described herein may or may not include a fluid. In some embodiments, the polymer compositions described herein include dry polyacrylamide (DPAM) polymer compositions.

When a polymer composition includes at least one type of fluid, the polymer composition may be referred to herein as a “well treatment fluid”. A well treatment fluid may include a fluid used in hydraulic fracturing that includes a copolymer as described herein. The copolymer may be present in a well treatment fluid at an amount of about 5 to about 1,000 ppm, about 5 to about 500 ppm, about 5 to about 250 ppm, or about 5 to about ppm.

In some embodiments, the fluid of a well treatment fluid includes water. In some embodiments, the fluid of a well treatment fluid includes a brine. The brine generally may have any concentration of salt, such as a salt concentration of about 10 ppm to about 300,000 ppm, such as about 1,000 to about 20,000 ppm. In some embodiments, the brine has a salt concentration of about 25,000 ppm to about 35,000 ppm.

The well treatment fluid, for example an aqueous treatment fluid, containing the tagged copolymers described herein, can be used in any well treatment fluid, including but not limited to stimulation, production and completion operations. For example, the well treatment fluid can be used for hydraulic fracturing applications or in an application where friction reduction is desired. Conventional fracturing fluids typically contain natural or synthetic water soluble polymers, which are well known in the art. Water soluble polymers viscosify the aqueous liquids at relatively low concentrations due to their high molecular weight.

In an exemplary embodiment, the treatment fluid comprises water and an exemplary emulsion described herein, such as an emulsion produced by the copolymerization methods described herein. The treatment fluids may be prepared by mixing an exemplary emulsion with water. The exemplary emulsion is a water-in-oil emulsion that undergoes rapid inversion upon addition to water. The speed at which the emulsion undergoes inversion can be measured by the amount of time required to substantially reach the maximum friction reduction.

The additional water that is mixed with the emulsion to form the treatment fluid may be freshwater, saltwater (e.g. water containing one or more salts dissolved therein), brine (e.g. produced from subterranean formations, or recycled water from the treatment process that contains additional salts), seawater, or combinations thereof. Generally, the water used may be from any source, provided that it does not contain an excess of compounds that may adversely affect other components in the aqueous treatment fluid or the formation itself. In certain exemplary embodiments, the water is brine with a total dissolved solids content (TDS) of about 40,000 to about 300,000 ppm, or about 100,000 to about 260,000 ppm. In certain exemplary embodiments, the total divalent cationic species content of the brine is in the range of about 0 to about 100,000 ppm, or about 1,000 to about 50,000 ppm.

Total dissolved solids (“TDS”) refers to the sum of all minerals, metals, cations, and anions dissolved in water. As most of the dissolved solids are typically salts, the amount of salt in water is often described by the concentration of total dissolved solids in the water.

Dissolved solids in typical water samples include soluble salts that yield ions such as sodium (Na⁺), calcium (Ca²⁺), magnesium (Mg²⁺), bicarbonate (HCO₃⁻), sulfate (SO₄⁻²), or chloride (Cl). Water that contains significant amounts of dissolved salts is sometimes broadly called saline water or brine, and is expressed as the amount (by weight) of TDS in water in mg/L. On average, seawater in the world's oceans has a salinity of about 3.5%, or 35 parts per thousand. More than 70 elements are dissolved in seawater, but only six elements make up greater than 99% by weight.

Total dissolved solids can be determined by evaporating a pre-filtered sample to dryness, and then finding the mass of the dry residue per liter of sample. A second method uses a Vernier Conductivity Probe to determine the ability of the dissolved salts in an unfiltered sample to conduct an electrical current. The conductivity is then converted to TDS. Either of these methods yields a TDS value, typically reported in units of mg/L.

Broadly speaking, either “saline water” or “brine” is often understood to be water containing any substantial concentration of dissolved inorganic salts, regardless of the particular concentration. The terms “saline water,” “brine,” and other terms regarding water may sometimes be used to refer to more precise ranges of concentrations of TDS. Although the specific ranges of TDS for various types of water are not universally agreed upon, various sources have used the definitions and ranges shown in the table below. As used herein, unless the context otherwise suggests, the terms for classifying water based on concentration of TDS will generally be understood as defined in the table below.

TABLE 1
Classification of Water Based on TDS Concentration

| Water | PPM | LG/Gal (US) |
|---|---|---|
| Potable | <250 | <0.0021 |
| Freshwater | <1,000 | <0.0083 |
| Brackish | 1,000-15,000 | 0.0083-0.0417 |
| Saline | 15,000-30,000 | 0.0417-0.1251 |
| Seawater | 30,000-40,000 | 0.1251-0.3338 |
| Brine | >40,000 | >0.3338 |

In exemplary embodiments, the tagged copolymers described herein may be present in the treatment fluid in an amount of about 0.010% to about 10% by weight of the treatment fluid.

In these applications, the treatment fluid, can be configured as a gelled fluid, such as a linear gel, a crosslinked gel, or a foamed gel fluid; acidic fluids, water and potassium chloride treatments, and the like. The treatment fluid may be injected at a pressure effective to create one or more fractures in the subterranean formation. Depending on the type of well treatment fluid utilized, various additives may also be added to the fracturing fluid to change the physical properties of the fluid or to serve a certain beneficial function. In one embodiment, the fluid does not contain a sufficient amount of water-soluble polymer to form a gel.

In exemplary embodiments, the well treatment fluid comprises a proppant.

In various exemplary embodiments, the proppants may be finely sized sand. Generally sand is referred to by the size of mesh which the sand will pass through, and the size of mesh which the sand will not pass through. Typically, a 20-40 mesh sand is used but other sizes, such as 40-50 or 40-60, may be utilized. Sand is also characterized by the“roundness” of the sand particles. Generally rounder sand is utilized in order to create more uniform void spaces between the particles and therefore better permeability within the propped fracture. Fracturing fluids also contain, for example, viscosifiers to slow the rate at which sand will separate from the fluids and permit the sand to be carried farther into the fractures.

In other exemplary embodiments, other types of proppants may be used. For example, the proppant may be a ceramic proppant. The proppant may be a coated proppant, such as proppants with coatings with low coefficients of friction in order to reduce erosion caused by the fracturing fluid. Coatings also may be used to make the sand particles more round. Examples of such coatings include antimony trioxide, bismuth, boric acid, calcium barium fluoride, copper, graphite, indium, fluoropolymers (FTFE), lead oxide, lead sulfide, molybdenum disulfide, niobium dielenide, polytetrafluoroethylene, silver, tin, or tungsten disulfide or zinc oxide. Ceramic proppants are suggested, for example, in U.S. Pat. No. 4,555,493 to Watson et al, and low density ceramic proppants are suggested in U.S. Pat. No. 8,420,578 to Usova et al..

Fracturing fluids, such as the well treatment fluids described herein, may also contain other components as necessary or desired. For example, the fracturing fluids may contain acids for breaking the thickening polymers, salts such as calcium chlorides to increase the density of the fluids, corrosion inhibitors or other additives in the fracturing fluids.

Also, fluid loss agents may be added to partially seal off the more porous sections of the formation so that the fracturing occurs in the less porous strata. Other oilfield additives that may be added to the fracturing fluid include emulsion breakers, antifoams, scale inhibitors, iron sulfide (FeS) and or 02 scavengers, biocides, crosslinking agents, surface tension reducers, buffers, fluorocarbon surfactants, clay stabilizers, fluid loss additives, foamers, friction reducers, temperature stabilizers, diverting agents, shale and clay stabilizers, paraffin/asphaltene inhibitors, corrosion inhibitors, and acids. For example, an acid may be included in the aqueous treatment fluids, among other things, for a matrix or fracture acidizing treatment. In fracturing embodiments, propping agent may be included in the aqueous treatment fluids to prevent the fracture from closing when the hydraulic pressure is released. In a particular embodiment, the treatment fluid further comprises a biocide.

### Methods of Synthesis

The compounds or isomers of formula (I), (I′), or (II) may be synthesized with any technique, including those provided herein.

In some embodiments, the compounds or isomers of formula (I), (I′), or (II) are formed via a condensation reaction. The condensation reaction may include contacting an aryl alcohol, a condensation catalyst, and a compound according to formula (A) to form the condensation product. The condensation product may include a compound or isomer of formula (I), (I′), or (II), which, as explained herein, includes the salts, hydrates, salt hydrates, stereoisomers, dehydrates, tautomers, and derivatives of the compounds or isomers of formula (I), (I′), or (II).

The compound according to Formula (A) has the following structure:

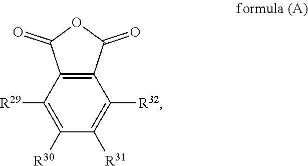

wherein R²⁹, R³⁰, R³¹, and R³² are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl.

In some embodiments, R²⁹, R³⁰, R³¹, and R³² are hydrogen.

As used herein, the phrase “aryl alcohol” generally refers to a compound that includes (i) an aryl moiety, and (ii) at least one hydroxyl moiety. In some embodiments, the aryl alcohol includes (i) an aryl moiety, and (ii) two hydroxyl moieties. In some embodiments, the aryl alcohol is resorcinol. In some embodiments, the aryl alcohol is 1,6-dihydroxynaphthalene. The aryl alcohol, however, may include any compound that is capable of forming a compound or isomer of formula (I), (I′), or (II). For example, when the methods provided herein are used to produce a compound or isomer of formula (I), the aryl alcohol may be of the following formula:

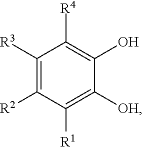

wherein R¹, R², R³, and R⁴ are as defined herein.

The contacting of the aryl alcohol, the condensation catalyst, and the compound of formula (A) may occur at any temperature and/or pressure that is effective to form the condensation product. In some embodiments, the contacting of the aryl alcohol, the condensation catalyst, and the compound of formula (A) occurs at a temperature of about 50° C. to about 150° C., about 75° C. to about 150° C., about 100° C. to about 150° C., or about 100° C. to about 125° C. In some embodiments, the contacting of the aryl alcohol, the condensation catalyst, and the compound of formula (A) occurs at ambient pressure, and a temperature of about 50° C. to about 150° C., about 75° C. to about 150° C., about 100° C. to about 150° C., or about 100° C. to about 125° C.

The condensation catalyst may include any catalyst capable of effecting the condensation of the aryl alcohol and the compound of formula (A). In some embodiments, the condensation catalyst is a Lewis acid. Non-limiting examples of Lewis acids include ZnCl₂, FeCl₃, AlCl₃, and BC₁₃. In some embodiments, the condensation catalyst is a sulfonic acid. The sulfonic acid may include an C₁-C₆ alkyl sulfonic acid, a C₅-C₁₄ aryl sulfonic acid, or a combination thereof. In some embodiments, the C₁-C₆ alkyl sulfonic acid is methanesulfonic acid (MeSO₃H). In some embodiments, the C₅-C₁₄ aryl sulfonic acid is p-toluenesulfonic acid.

Also provided herein are methods of polymerizing a compound or isomer of formula (I), (I′), or (II). In some embodiments, the methods include contacting a compound or isomer of formula (I), (I′), or (II)(e.g., a condensation product of the foregoing methods) with at least one second monomer to form a copolymer, wherein the at least one second monomer includes a polymerizable double bond or triple bond. The at least one second monomer may be contacted with an amount of the condensation product effective to produce a copolymer that includes a desirable amount of the condensation product as described herein, for example, any of the amounts recited herein, such as about 0.01% to about 5%, or about 0.01% to about 2%, by weight, based on the weight of the copolymer.

The at least one second monomer generally may include any monomer that is polymerizable due to the presence of a polymerizable double bond or triple bond. The phrases “polymerizable double bond”, “polymerizable triple bond”, and the like refer to bonds that may react with a functional group of at least one other monomer (e.g., under conditions described herein) to form a polymer. In some embodiments, the at least one second monomer includes sodium acrylate, acrylamide, or a combination thereof.

The polymer compositions provided herein generally may be prepared by any polymerization method. For example, a free-radical polymerization method may be employed. Other exemplary methods include aqueous bulk/dispersion polymerization, solution polymerization, or emulsion polymerization. In some embodiments, the polymerization process is a solution polymerization, wherein water is charged to a reaction vessel fitted with a mechanical stirrer and water condenser, and heated to a temperature within a range of about 45° C. to about 1500° C., or about 45° C. to about 110° C. One or more polymerization initiators may be added to the reactor. The choice of initiator may inform the temperature at which the reaction is performed. A first monomer may be added to the reactor, added to a monomer feed or fed separately. A monomer feed(s), soluble initiator feed, and optionally a chain transfer reagent feed may be added to a vessel at a predetermined time or over time.

In some embodiments, the method of polymerization includes an emulsion polymerization. In some embodiments, the methods include providing an aqueous phase having a first pH, the aqueous phase comprising water and at least one second monomer, as described herein. The at last one second monomer may include any of those described herein. In some embodiments, the at least one second monomer includes acrylamide, acrylic acid, acrylamide-2-methylpropanesulfonic acid, or a combination thereof.

The pH of the aqueous phase may be adjusted if needed or desirable. For example, the pH of the aqueous phase may be adjusted from a first pH to a second pH, wherein the second pH is less than or greater than the first pH. An alkaline liquid may be used to adjust the pH of the aqueous phase.

A first monomer, as described herein, may be disposed in the aqueous phase. The first monomer may be disposed in the aqueous phase after the optional pH adjustment, but other embodiments are envisioned.

The aqueous phase generally may include about 95 to 65% by weight of the initial emulsion. Preferably, it may include about 80 to 70% thereof. In addition to water, the aqueous phase contains the monomers being polymerized, generally in an amount of less than about 50%, about 15 to about 40%, or about 22 to about 35%, by weight of the total emulsion, and generally chain transfer agents, initiators and sequesterants. Alternatively, the chain transfer agents, initiators and sequesterants may be added to the system after the preliminary emulsion has been prepared. The initiator may also be added continuously during the polymerization to control the rate of polymerization depending upon the particular monomers used and their reactivities.

The methods of polymerization also include providing an oil phase. The oil phase may include an oil and at least one emulsifying surfactant.

In some embodiments, the aqueous phase is combined with the oil phase to form an emulsion. The aqueous phase and the oil phase may be combined in any order and in any manner. In some embodiments, the combining of the aqueous phase and the oil phase includes homogenizing the aqueous phase and the oil phase. A homogenizing mixer may be used for this purpose.

The oil phase of the emulsion, which may include from about 5% to about 35% by weight of the emulsion, may include one or more inert hydrophobic liquids. In some embodiments, the oil phase comprises about 20% to about 30% by weight of the emulsion. The oil used may be selected from a large class of organic liquids which are immiscible with water, including liquid hydrocarbons and substituted liquid hydrocarbons. Representative examples of such oils include benzene, xylene, toluene, mineral oils, kerosenes, naphthas, chlorinated hydrocarbons, such as perchloroethylene, and the like.

The oil phase may contain one or more primary or emulsifying surfactants, i.e. conventional emulsion polymerization stabilizers. Such stabilizers are well known to the art to promote the formation and stabilization of water-in-oil emulsions. Normally such emulsifiers have HLB values in the range of about 2 to about 10, preferably less than about 7. Suitable such emulsifiers include the sorbitan esters, phthalic esters, fatty acid glycerides, glycerine esters, as well as the ethoxylated versions of the above and any other well-known relatively low HLB emulsifier. Examples of such compounds include sorbitan monooleate, the reaction product of oleic acid with isopropanolamide, hexadecyl sodium phthalate, decyl sodium phthalate, sorbitan stearate, ricinoleic acid, hydrogenated ricinoleic acid, glyceride monoester of lauric acid, glyceride monoester of stearic acid, glycerol diester of oleic acid, glycerol triester of 12-hydroxystearic acid, glycerol triester of ricinoleic acid, and the ethoxylated versions thereof containing 1 to 10 moles of ethylene oxide per mole of the basic emulsifier. Thus, any emulsifier may be utilized which will permit the formation of the initial emulsion and stabilize the emulsion during the polymerization reaction.

These primary surfactants may be used alone or in mixtures and are utilized in amounts of not greater than about 5%, about 4%, about 3%, about 2% or about 1% by weight of the total emulsion.

The emulsion formed by combining the aqueous phase and the oil phase may optionally be deoxygenized. The deoxygenizing of the emulsion may include sparging the emulsion with an inert gas, such as N₂.

In some embodiments, an initiator is disposed in the emulsion. The initiator may include any of those described herein. For example, the initiator may include a redox couple. Typically redox initiators include a reducing agent such as sodium sulfite, sodium metabisulphite, or sulfur dioxide and oxidizing compounds such as ammonium persulphate or a peroxy compound, such as t-butyl hydroperoxide. Non-limiting examples of redox initiators are described in U.S. Patent Application Publication No. 2019/0241794, which is incorporated by reference herein.

Before, during, or after an initiator is disposed in the emulsion, the emulsion may be contacted with an initiator, such as a gas, to copolymerize the first monomer and the at least one second monomer to form a copolymer as described herein.

Suitable emulsion polymerization techniques may have a variety of different initiation temperatures depending on, among other things, the amount and type of initiator used, the amount and type of monomers used, and a number of other factors known to those of ordinary skill in the art. In one embodiment, a suitable emulsion polymerization technique may have an initiation temperature of about 25° C. Due to the exothermic nature of the polymerization reaction, the mixture may be maintained at a higher temperature than the initiation temperature during procession of the polymerization reaction, for example, in the range of from about 30° C. to about 70° C., or from about 40° C. to about 60° C.

The method also may include separating a copolymer from unreacted monomers. This may include removing unreacted monomers from the emulsion. The removal of the unreacted monomers from the emulsion may be achieved by any known technique. For example, the unreacted monomers may be removed by heating the emulsion to a temperature effective to achieve the removal of the monomers.

In some embodiments, the methods include disposing in the emulsion an inverting surfactant package.

In some embodiments, the emulsion that includes a copolymer is filtered. Any suitable filter may be used for this purpose.

The initiator may be any free radical producing material well known in the art. The preferred free radical initiators are the redox-type and the azo-type polymerization initiators and they are generally used in an amount of about 0.0005 to 0.5 percent by weight of the total emulsion. Radiation may also be used to initiate the reaction.

In some embodiments, the polymerization of monomers, including at least one first monomer and at least one second monomer, is achieved in the presence of one or more polymerization initiators. The polymerization initiators may include a redox couple. The polymerization initiator may include inorganic peroxides, for example ammonium persulfate (APS), hydroxymethanesulfinic acid monosodium salt dehydrate, potassium persulfate, and sodium persulfate; organic peroxides, for example tert-butyl hydroperoxide (TBHP), tert-butyl peracetate, cumene hydroperoxide, 2,5-Di(tert-butylperoxy)-2,5-dimethyl-3-hexyne, dicumyl peroxide, 2,5-bis(tert-butylperoxy)-2,5-dimethylhexane, 2,4-pentanedione peroxide, 1,1-bis(tert-butylperoxy)-3,3,5-trimethylcyclohexane, 1,1-bis(tert-butylperoxy)cyclohexane, 1,1-bis(tert-amylperoxy)cyclohexane, benzoyl peroxide, 2-butanone peroxide, tert-butyl peroxide, lauroyl peroxide, tert-butyl peroxybenzoate, and tert-butylperoxy 2-ethylhexyl carbonate; azo compounds, for example azobisisobutyronitrile (AIBN), 4,4′-azobis(4-cyanovaleric acid), 1,1′-azobis(cyclohexanecarbonitrile), 2,2′-azobis(2-methylpropionamidine) dihydrochloride, and 2,2′-azobis(2-methylpropionitrile); tetrakis(hydroxymethyl)phosphonium sulfate (THPS); cerium ammonium nitrate; perchlorates; triphenylphosphine; and the like, and compositions or mixtures including one or more of these initiators. In some embodiments, the initiator is selected from the group consisting of ammonium persulfate, tert-butyl hydroperoxide, and 4,4′-azobis(4-cyanovaleric acid).

Polymerization initiators generally may be used at an amount of about 0.01% to about 10%, by weight, based on the total weight of the monomers. Polymerization initiators may be used in conjunction with heat to initiate polymerization of monomers. In some embodiments, two or more initiators are used; for example, an inorganic peroxide and an organic peroxide, and/or a redox couple. In some embodiments, ammonium persulfate (APS) and an organic peroxide are used to initiate polymerization. The initiator or initiators used to achieve polymerization may affect the physical properties of the resulting polymer. The initiator or initiators may be added to a polymerization reaction mixture, for example, at the start of the reaction, at various times during the polymerization, and/or gradually over time, e.g., over several minutes or hours. If two or more initiators are used, then the initiators may be dosed simultaneously or sequentially during polymerization. In some embodiments, one initiator is dosed at the start of polymerization, at various times during polymerization, and/or gradually over time, and a different initiator is used at later stages the polymerization.

Any conventional chain transfer agent may be employed, such as propylene glycol, isopropanol, 2-mercaptoethanol, sodium hypophosphite, dodecyl mercaptan and thioglycolic acid. The chain transfer agent is generally present in an amount of about 0.01% to 10% by weight of the total emulsion, though more may be used.

Any conventional sequesterant may also be present in the aqueous phase, such as ethylenediaminetetraacetic acid or pentasodium diethylenetriamine pentaacetate. The sequesterant is generally present in an amount of about 0.01% to 2% by weight of the total emulsion, though more may be utilized.

Following preparation of the preliminary emulsion, polymerization of the monomers is commenced at a temperature sufficiently high to break down the initiator to produce the desired free radicals. Generally a suitable temperature is about −20° C. to about 200° C., or about 20° C. to 100° C.

Preferably the polymerization is run at a pH of about 2 to 12 and a suitable amount of base or acid may be added to the preliminary emulsion to achieve the desired pH. The polymerization is usually completed in about an hour or two to several days, depending upon the monomers employed and other reaction variables. It is generally carried out at atmospheric pressure, but higher pressures are advantageously used when volatile ingredients are involved.

In certain exemplary embodiments, once polymerization is complete, the amount of water in the emulsion may be reduced or removed as desired. For example, the water can be removed to a level of less than about 12%, or less than about 10%, or less than about 7%, or less than about 5%, or less than about 3% by weight. In exemplary embodiments, the removal of water is carried out by any suitable means, for example, at reduced pressure, e.g. at a pressure of about 0.00 to about 0.5 bars, or about 0.05 to about 0.25 bars. The temperature for water removal steps may typically be from about 50° C. to about 150° C., although techniques which remove water at higher temperatures may be used.

Following completion of the polymerization, the pH of the emulsion may be adjusted as desired. For an anionic polymer emulsion, the pH is generally about 4 to 10. A breaker or inverting surfactant, or blend of inverting surfactants, is generally added to yield a single package of final product. An “inverting surfactant package” may include one or more inverting surfactants. In exemplary embodiments, an inverting surfactant composition, as described herein, is added to the polymer emulsion. Other suitable breaker or inverting surfactants may be used in combination with the exemplary polymer and exemplary inverting surfactant composition in the emulsion. As described herein, the total amount of inverting surfactants present in the emulsion is about 1% to about 5% by weight, based on the total emulsion.

Once prepared, the emulsions of the present embodiments may be chemically modified in any known manner. “Chemically modified” is intended to cover further treatment of the dispersed water-soluble polymer and/or the addition of components to the dispersed water-soluble polymer which, without the stabilization provided by the emulsion stabilizers, would cause the normally water-soluble polymeric particles to coagulate or agglomerate. Examples of such further treatments are disclosed in U.S. Pat. Nos. 4,052,353 and 4,171,296, the disclosures of which are incorporated herein by reference. The emulsion of the present embodiments may also be concentrated in any suitable manner, such as is disclosed in U.S. Pat. No. 4,021,399, the disclosures of which is incorporated herein by reference.

A variety of different mixtures may be used to prepare an emulsion for use in the present embodiments. Suitable mixtures may include acrylamide, other monomers, water, a water-immiscible liquid, an initiator, and an emulsifier. Generally the one or more ethoxylated alcohol compounds can be combined with one or more inverting surfactants to form the inverting surfactant composition. The inverting surfactant composition can be added to the polymer emulsion to form a mixture. Optionally, the mixture may further comprise a base (e.g., sodium hydroxide) to neutralize the monomers in acid form such that the salt of the monomer is not formed, a complexing agent to allow the gradual release of monomers in the polymerization reaction, an activator to initiate polymerization at a lower temperature, and an inverter. Those of ordinary skill in the art, with the benefit of this disclosure, will, know the amount and type of components to include in the mixture based on a variety of factors, including the desired molecular weight and composition of the polymer and the desired initiation temperature.

Generally, the exemplary emulsions are particularly suitable for use in brine. The exemplary emulsions may be used in a range of temperatures, for example from about 5° C. to about 180° C., about 5° C. to about 99° C., or about 50° C. to about 95° C.

In certain exemplary embodiments, the emulsion may be used in combination with a proppant.

### Methods for Determining Polymer Concentration

Methods for determining polymer concentration are provided. In some embodiments, the methods include providing a system that includes a fluid in circulation, wherein the fluid includes a polymer composition as described herein; measuring with an analytical technique an amount of the first monomer in the system or the fluid to determine an amount of the polymer composition in the system or the fluid, wherein the measuring is performed periodically or continuously.

As used herein, the phrase “amount of the first monomer”, the phrase “amount of the polymer composition”, and the like refer to and include (i) an actual numerical amount (e.g., X grams) of the first monomer or the polymer composition, respectively, in a fluid or system, or (ii) a concentration (e.g., X ppm) of the first monomer or polymer composition, respectively, in a fluid or system.

In some embodiments, the methods include (a) adding to a system or fluid a predetermined amount of a polymer composition as described herein; (b) periodically or continuously measuring the amount of tagging units (i.e., first monomer) in the system or fluid to determine an amount of the polymer composition in the system or fluid; and (c) periodically or continuously further adding more or removing a portion of the polymer composition to or from the system or fluid when the measured amount of tagging units (i.e., first monomer) is less than or greater than, respectively, a predetermined value.

In some embodiments, when the measured amount of a tagging first monomer (or the polymer composition) in a system or fluid being treated is less than a predetermined value, more polymer composition may be added to the system or fluid. The predetermined value of polymer may be any amount necessary or desired for the particular system or fluid being treated.

The polymer compositions herein may be detected (e.g., measured) by any appropriate method, including, but not limited to, fluorescence spectroscopy. In some embodiments, the polymer compositions are detected with fixed wavelength fluorescence spectroscopy. Detection may be at the polymer maxima excitation (ex) and emission (em) wavelengths. These wavelengths may be determined using a scanning fluorometer in scanning mode. The level of fluorescence may be determined by the Beer-Lambert Law. For example, concentrations may be assigned by comparison of the emission intensity of a polymer composition sample with a calibration plot obtained from polymer samples of a known concentration. Any detection method which utilizes the fluorescence properties of the polymer compositions, particularly the first monomer, may be used, as necessary or desired.

The constituents of a liquid, e.g., water, may be considered when determining the proper application of the polymer compositions provided herein, as some of the constituents may have natural fluorescence properties (for example, certain polycyclic hydrocarbons) that may interfere with the detection of the tagging units (i.e., first monomer) of the polymer compositions. The chemical properties of produced water may vary considerably depending on the location and the geological formation of an oil field, as well as the type of hydrocarbons being produced. Produced water properties also may vary throughout the lifetime of a reservoir. Most of the naturally fluorescent properties of produced waters typically originate from hydrocarbon residues or other production chemicals in the produced waters. Even though the amount of these species might be minimal, fluorescence can detect these species at very low ppm levels.

In some embodiments, the polymer compositions can be used in combination or alternation with other tagged polymers or tagged polymer compositions, and in particular, with other tagged polymers or tagged polymer compositions including fluorescent moieties that have excitation and/or emission that are different from those of the polymer compositions described herein. The use of the polymer compositions described herein with other tagged polymers or tagged polymer compositions may be referred to as a multi-tagged system. A multi-tagged system could be used, for example, to allow an operator to monitor two different polymers in a system being treated with a polymer composition as provided herein. An example of such a system would include one in which more than one well is drilled and the oil from all wells is collected from one central location. A different polymer composition and/or other tagged polymer may be introduced to each well. From a single sample collected at the central location, an operator may determine which specific well requires more polymer composition and/or other tagged polymer by monitoring the presence and/or concentration of each tagged polymer.

In some embodiments, the polymer compositions are combined in a multi-tagged system with one or more polymers having a different tagging unit than the polymer compositions. Exemplary tagging units are described, for example, in one or more of the following references (each of which is incorporated herein by reference): U.S. Pat. Nos. 7,703,516; 7,943,058; 9,902,904; EP 1 636 142; EP 1 639 228; U.S. Patent Application Publication. No. 2012/0032093.

As used herein, the phrase “effective detection amount” refers to an amount of tagging units (i.e., first monomer) sufficient to provide suitable detection in a particular application. In some embodiments, the polymer composition includes an effective detection amount of tagging units (i.e., first monomer). In some embodiments, an effective detection amount of the first monomer in the polymer compositions is about 0.01% to about 30%, about 0.01% to about 20%, about 0.01% to about 15%, about 0.01% to about 10%; about 0.01% to about 8%; about 0.01% to about 7%; about 0.01% to about 5%; about 0.01% to about 3%, about 0.01% to about 2%, about 0.01% to about 1.5%, about 0.01% to about 1%, about 0.01% to about 0.75%, or about 0.01% to about 0.5%, by weight, based on the total weight of the polymer composition. An effective detection amount may be achieved by the amount of first monomer that is polymerized with an at least one second monomer to form a copolymer, an amount of unpolymerized first monomer added to a polymer composition, or a combination thereof.

The compounds and polymer compositions described herein may be used in a variety of systems, including aqueous systems. Non-limiting examples of such systems include boiler water, geothermal water, heat district water, cooling water, seawater (e.g., in oil platform applications), brackish water, oilfield water (e.g., topside and/or downhole), enhanced oil recovery, fracturing work, oil sand tailing treatment, mining processes, tailing treatment, municipal water treatment plant, and industrial water treatment plant. The amount of polymer composition that is effective to achieve a desired purpose in a particular aqueous system may be determined by routine experimentation in light of the guidance provided herein. The amount of polymer composition added to the aqueous system may vary over a relatively broad range, depending on the nature of the aqueous system and the type of scale. For example, the amount of polymer added to the aqueous system may be in the range of about 0.1 part per million to about 50,000 parts per million, about 0.1 part per million to about 25,000 parts per million, about 0.1 part per million to about 10,000 parts per million, about 0.1 part per million to about 1,000 parts per million, about 0.1 part per million to about 500 parts per million, or about 100 parts per million to about 200 parts per million, based on the capacity of the aqueous system.

### Methods of Hydraulic Fracturing

Also provided herein are methods of hydraulic fracturing, which, in some instances, rely in whole or in part on various “Methods for Determining Polymer Concentration” described herein. For example, any of the instruments, analytical techniques, concentrations, etc. of the “Methods for Determining Polymer Concentration” may be independently incorporated into the methods of hydraulic fracturing described herein.

In some embodiments, the methods of hydraulic fracturing include providing a well treatment fluid (i.e., any polymer composition described herein that includes a fluid); disposing the well treatment fluid in a well of a reservoir; and measuring with an analytical technique an amount of the first monomer in the well treatment fluid after the disposing of the well treatment fluid in the well to (i) identify one or more producing stages, (ii) determine an optimum concentration of the copolymer in the well treatment fluid, or (iii) determine whether the well treatment fluid is recyclable.

If an optimum concentration of the copolymer in the well treatment fluid is determined, then well treatment fluids used in the future, or additional amounts of well treatment fluid used in the well may include the optimum amount of the copolymer.

If the well treatment fluid is recyclable, then the well treatment fluid may be disposed in the same well a second time, or a different well.

In some embodiments, the methods of hydraulic fracturing include providing a first well treatment fluid as described herein (i.e., any polymer composition that includes a fluid); providing a second well treatment fluid as described herein, wherein (A) a peak fluorescence emission of the first monomer of the first well treatment fluid and (B) a peak fluorescence emission of the first monomer of the second well treatment fluid occur at different wavelengths; disposing the first well treatment fluid in a first well of a reservoir; disposing the second well treatment fluid in a second well of the reservoir; and measuring with an analytical technique (i) an amount of the first monomer of the first well treatment fluid and (ii) an amount of the first monomer of the second well treatment fluid after the disposing of the first and the second well treatment fluids in the first and the second wells, respectively, to determine (a) a degree of connectivity between the first well and the second well of the reservoir, or (b) one or more producing stages of the first well or the second well.

The peak fluorescence emission of the first monomer of the first well treatment fluid and the peak fluorescence emission of the first monomer of the second well treatment fluid may differ to any extent. The difference may be effective to ease the distinct measurement of the two different first monomers. The peak fluorescence emission(s) also may differ to any desired extent from the peak fluorescence emission of any other components that may be present in a well treatment fluid.

The analytical technique may be performed at any point after the one or more two well treatment fluids are disposed in a well. In some embodiments, the measuring with the analytical technique occurs during flow back operations.

### Embodiments

The following is a listing of non-limiting embodiments: Embodiment 1. A polymer composition comprising a copolymer comprising (i) a first monomer comprising (a) a compound of formula (I), formula (I′), or formula (I″), or (b) a compound or an isomer of formula (II), wherein the first monomer is a tagging monomer, and (ii) at least one second monomer comprising at least one polymerizable double bond or at least one polymerizable triple bond;

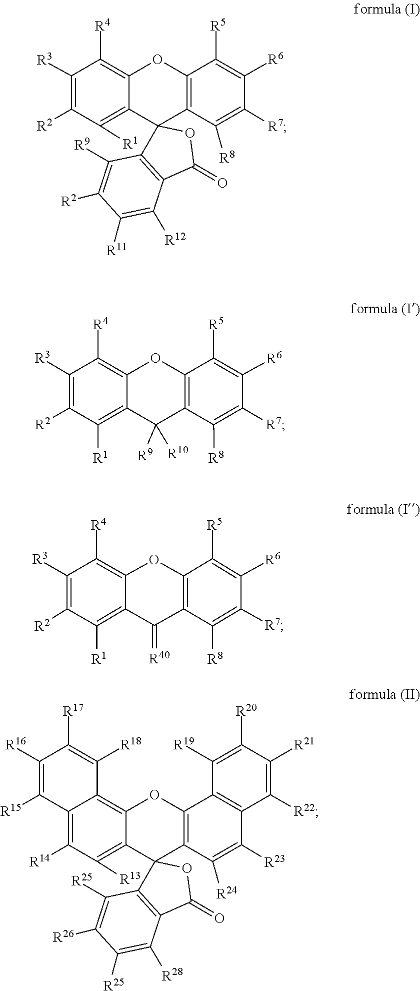

wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R², R²¹,R², R²³, R²⁴, R²⁵, R²⁶, R²⁷, and R²⁸ are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl,
wherein R⁴° is selected from the group consisting of C₁-C₆ alkoxy, C₂-C₆ alkenoxy, C₂-C₆ alkynoxy, —N(R′), —N(R′)(R″), C₂-C₆ alkyl, C₂-C₆ alkenyl, C₂-C₆ alkynyl, and C₄-C₁₄ aryl,
wherein R′ and R″ are independently selected from the group consisting of hydrogen and C₁-C₆ alkyl, and
wherein the isomers of Formula (II) comprise a compound of Formula (IIi), a compound of Formula (IIii), a compound of Formula (IIiii), or a combination thereof—

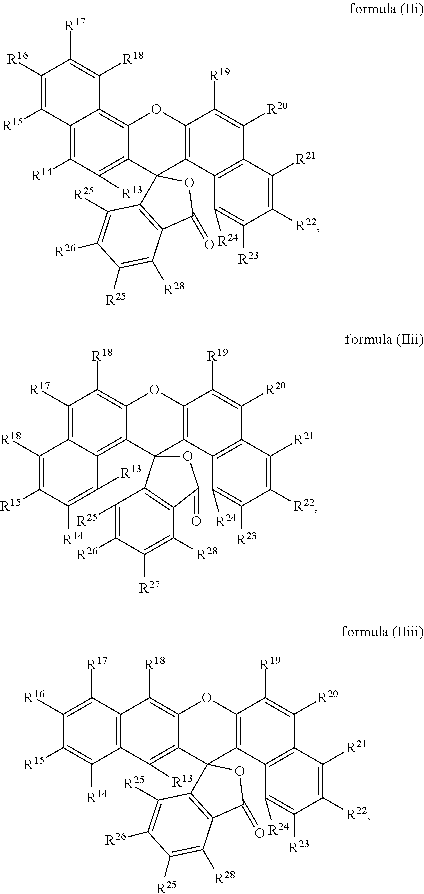

Embodiment 2. The polymer composition of Embodiment 1, wherein the copolymer has a weight average molecular weight (M_{W}) of (i) greater than 1,000,000 Da, or (ii) about 7,500,000 to about 30,000,000 Da.

Embodiment 3. The polymer composition of any of the preceding Embodiments, wherein the first monomer is present in the copolymer at an amount of about 0.005% to about 5%, by weight, based on the weight of the copolymer, about 0.01% to about 2%, by weight, based on the weight of the copolymer, or about 0.01% to about 1%, by weight, based on the weight of the copolymer.

Embodiment 4. The polymer composition of any of the preceding Embodiments, wherein the at least one second monomer comprises acrylamide, sodium acrylate, acrylic acid, sodium acrylamido-2-methylpropanesulfonic acid, or a combination thereof.

Embodiment 5. The polymer composition of any of the preceding Embodiments, wherein the first monomer comprises the compound of formula (I), wherein R¹, R², R⁴, R⁵, R⁷, R⁸, R⁹, R¹⁰, R¹¹, and R¹² are hydrogen, or formula (I′) or (I″), wherein R¹, R², R⁴, R⁵, R⁷, and R⁸ are hydrogen.

Embodiment 6. The polymer composition of any of the preceding Embodiments, wherein R³ and R⁶ are hydroxyl.

Embodiment 7. The polymer composition of any of the preceding Embodiments, wherein R³ and R⁶ are —N(R′)(R″).

Embodiment 8. The polymer composition of Embodiment 7, wherein R′ and R″ are independently selected from hydrogen and an unsubstituted C₁-C₆ alkyl.

Embodiment 9. The polymer composition of any of the preceding Embodiments, wherein the first monomer comprises the compound or isomer of formula (II), wherein R¹³, R¹⁴, R¹⁵, R¹⁷, R¹⁸, R¹⁹, R²⁰, R²², R²³, R²⁴, R²⁵, R²⁶, R²⁷, and R²⁸ are hydrogen.

Embodiment 10. The polymer composition of any of the preceding Embodiments, wherein the first monomer comprises the compound or isomer of formula (II), wherein R¹⁶ and R²¹ are hydroxyl.

Embodiment 11. The polymer composition of any of the preceding Embodiments, wherein R¹⁶ and R²¹ are —N(R′)(R″).

Embodiment 12. The polymer composition of Embodiment 11, wherein R′ and R″ are independently selected from hydrogen and an unsubstituted C₁-C₆ alkyl.

Embodiment 13. The polymer composition of any one of Embodiments 1 to 12, wherein the copolymer is an anionic copolymer.

Embodiment 14. The polymer composition of any one of Embodiments 1 to 13, wherein the first monomer is an end group of the copolymer.

Embodiment 15. The polymer composition of any one of Embodiments 1 to 13, wherein the first monomer is incorporated into the backbone of the copolymer.

Embodiment 16. A well treatment fluid comprising the copolymer of any one of Embodiments 1 to 14.

Embodiment 17. The well treatment fluid of Embodiment 16, wherein the copolymer is present in the well treatment fluid at an amount of about 10 to about 1,000 ppm, about 10 to about 500 ppm, about 10 to about 250 ppm, or about 10 to about 200 ppm.

Embodiment 18. The well treatment fluid of Embodiment 16 or 17, wherein the well treatment fluid comprises water.

Embodiment 19. The well treatment fluid of Embodiment 16 or 17, wherein the well treatment fluid comprises brine.

Embodiment 20. The well treatment fluid of Embodiment 19, wherein the brine has a salt concentration of about 10 ppm to about 300,000 ppm.

Embodiment 21. The well treatment fluid of Embodiment 19, wherein the brine has a salt concentration of about 25,000 ppm to about 35,000 ppm.

Embodiment 22. A method of hydraulic fracturing, the method comprising providing a well treatment fluid of any one of Embodiments 16 to 21; disposing the well treatment fluid in a well of a reservoir; and measuring with an analytical technique an amount of the first monomer in the well treatment fluid after the disposing of the well treatment fluid in the well to (i) identify one or more producing stages, (ii) determine an optimum concentration of the copolymer in the well treatment fluid, or (iii) determine whether the well treatment fluid is recyclable.

Embodiment 23. A method of hydraulic fracturing, the method comprising providing a first well treatment fluid of any one of Embodiments 16 to 21; providing a second well treatment fluid of any one of Embodiments 16 to 21, wherein (A) a peak fluorescence emission of the first monomer of the first well treatment fluid and (B) a peak fluorescence emission of the first monomer of the second well treatment fluid occur at different wavelengths; disposing the first well treatment fluid in a first well of a reservoir; disposing the second well treatment fluid in a second well of the reservoir; and measuring with an analytical technique (i) an amount of the first monomer of the first well treatment fluid and (ii) an amount of the first monomer of the second well treatment fluid after the disposing of the first and the second well treatment fluids in the first and the second wells, respectively, to determine (a) a degree of connectivity between the first well and the second well of the reservoir, or (b) one or more producing stages of the first well or the second well.

Embodiment 24. The method of Embodiment 22 or 23, wherein the measuring with the analytical technique occurs during flow back operations.

Embodiment 25. The method of any one of Embodiments 22 to 24, wherein the analytical technique comprises fluorescence spectroscopy.

Embodiment 26. A method of polymerization, the method comprising providing an aqueous phase having a first pH, the aqueous phase comprising water and at least one second monomer of Embodiment 1; optionally adjusting the first pH to a second pH; disposing a first monomer of Embodiment 1 in the aqueous phase; providing an oil phase comprising an oil and at least one emulsifying surfactant; combining the aqueous phase and the oil phase to form an emulsion; optionally deoxygenizing the emulsion; disposing an initiator in the emulsion; and contacting the emulsion with an initiator to copolymerize the first monomer and the at least one second monomer to form the copolymer of any one of Embodiments 1 to 14 in the emulsion.

Embodiment 27. The method of Embodiment 26, further comprising removing unreacted monomers from the emulsion.

Embodiment 28. The method of Embodiment 26 or 27, further comprising disposing in the emulsion an inverting surfactant package.

Embodiment 29. The method of any one of Embodiments 26 to 28, further comprising filtering the emulsion comprising the copolymer.

Embodiment 30. The method of any one of Embodiments 26 to 29, wherein the at least one second monomer comprises acrylamide, acrylic acid, sodium acrylamido-2-methyl-propanesulfonic acid, or a combination thereof.

Embodiment 31. The method of any one of Embodiments 26 to 30, wherein the second pH is less than the first pH.

Embodiment 32. The method of any one of Embodiments 26 to 31, wherein the adjusting of the first pH comprises contacting the aqueous phase with an alkaline liquid.

Embodiment 33. The method of any one of Embodiments 26 to 32, wherein the combining of the aqueous phase and the oil phase comprises homogenizing the aqueous phase and the oil phase.

Embodiment 34. The method of any one of Embodiments 26 to 33, wherein the deoxygenizing of the emulsion comprises sparging the emulsion with an inert gas.

Embodiment 35. The method of any one of Embodiments 26 to 34, wherein the initiator comprises a redox couple.

Embodiment 36. The method of any one of Embodiments 26 to 34, wherein the initiator comprises an organic hydroperoxide.

Embodiment 37. The method of any one of Embodiments 26 to 36, wherein the initiator further comprises a gas having a flow rate, and the method further comprises modifying the flow rate to control a rate of copolymerization.

Embodiment 38. The method of any one of Embodiments 26 to 37, wherein the emulsion, after the copolymerization, is a water-in-oil emulsion.

Embodiment 39. The method of Embodiment 38, further comprising disposing the water-in-oil emulsion in an aqueous fluid to form a well treatment fluid.

Embodiment 40. The polymer composition or methods of any one of Embodiments 1 to 39, wherein the first monomer exhibits a fluorescence emission maximum at about 410 nm to about 680 nm, about 410 nm to about 600 nm, about 410 nm to about 590 nm, about 410 nm to about 520 nm, about 410 nm to about 500 nm, about 440 nm to about 450 nm, about nm to about 520 nm, about 550 nm to about 590 nm, about 640 nm to about 680 nm, or about 570 nm to about 600 nm.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of various embodiments, applicants in no way disclaim these technical aspects, and it is contemplated that the present disclosure may encompass one or more of the conventional technical aspects discussed herein.

The present disclosure may address one or more of the problems and deficiencies of known methods and processes. However, it is contemplated that various embodiments may prove useful in addressing other problems and deficiencies in a number of technical areas. Therefore, the present disclosure should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

In the descriptions provided herein, the terms “includes,” “is,” “containing,” “having,” and “comprises” are used in an open-ended fashion, and thus should be interpreted to mean “including, but not limited to.” When compositions, systems, or methods are claimed or described in terms of “comprising” various steps or components, the compositions, systems, or methods can also “consist essentially of” or “consist of” the various steps or components, unless stated otherwise.

The terms “a,” “an,” and “the” are intended to include plural alternatives, e.g., at least one. For instance, the disclosure of “a first monomer”, “a polymer composition”, and the like, is meant to encompass one, or mixtures or combinations of more than one first monomer, polymer composition, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in some embodiments, the compounds exhibit fluorescence emission maxima at about 490 nm to about 500 nm. This range should be interpreted as encompassing emission maxima of about 490 nm and about nm, and further encompasses “about” each of 491 nm, 492 nm, 493 nm, 494 nm, 495 nm, nm, 497 nm, 498 nm, and 499 nm, including any ranges and sub-ranges between any of these values.

As used herein, the term “about” means plus or minus 10% of the numerical value of the number with which it is being used.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

In the following examples, fluorescein was copolymerized with polyacrylamide and sodium acrylate to make EPAM. The resulted polymer exhibited a standard viscosity (SV) similar to that of a non-tagged polymer (Table 1 below). The analytical results confirmed that the tag was polymerized and attached to the polymer backbone. The tag attachment to the main polymer chain likely occurred through chain transfer reactions.

The detectability of the polymers was verified in synthetic brine and later validated with produced water received from a field. Although many produced waters can contain fluorescing interferences, the emission wavelength was optimizable to reduce interferences, and simplify the sample treatment prior to measurement. Application data (friction reducer capability) of both tagged and non-tagged polymer were similar, indicating that tagging had little or no adverse effect on the applicability of the polymer.

### Example 1—Preparing of an Embodiment of a Tagged Polymer

In this example, fluorescein was copolymerized with polyacrylamide and sodium acrylate (APAM).

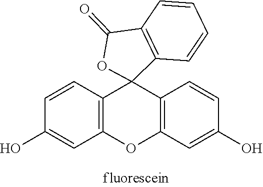 [SMILES: O=C1OC2(c3ccc(O)cc3Oc3cc(O)ccc32)c2ccccc21]

The fluorescein tagged polymers of this example were prepared by using a reverse emulsion polymerization technique.

First, a water phase was prepared by mixing together acrylamide, acrylic acid, and water. Then the pH was adjusted to a pH of about 4 to about 8. After the pH adjustment, fluorescein and other additives were added to the water phase.

An oil phase was prepared by mixing oil and emulsifying surfactants. The oil phase and the water phase were combined and homogenized.

The resulting emulsion was transferred to a jacketed glass reactor equipped with an overhead stirrer and gas tubing. The emulsion was deoxygenized by sparging with nitrogen gas.

A redox initiator added to the emulsion to initiate polymerization. After the polymerization stage, the residual monomers were burned off, and an inverting surfactant package was added to the emulsion while stirring.

Finally, the product was filtered through the filter cloth. The recipes of the tagged emulsions were based on two commercial available products: “Product 1” was a high viscosity friction reducer (HVFR) that is anionic, and provides effective HVFR performance in light brines at low dosages; and “Product 2” was an emulsion friction reducer that is anionic, and configured for fresh water applications.

Tagged polymers prepared by this example were fluorescent polymers (excitation max ˜490-500 nm, emission max ˜510-520 nm, fluorescent active in from neutral to alkali).

### Example 2 Analysis of Tagged Polymers

The tagged polymers of Example 1 exhibited SVs (serum viscosity) in a range similar to untagged polymer for Product 2, however, the differences between tagged and untagged polymers were about 1-1.5 cP for Product 1 (Table 1).

TABLE 2
EPAM polymerization results (tagged and not-tagged)

| | | Tag | | |
|---|---|---|---|---|
| Sample | Product | (monomer wt %) | IPA, ppm | SV (cP) |
| 1 | Product 2 | 0 | 0 | 7.46 |
| 2 | Product 2 | 0.01 | 0 | 7.45 |
| 3 | Product 1 | 0 | 2481 | 8.15 |
| 4 | Product 1 | 0.01 | 1000 | 6.9 |

SV of Product 1 decreased further by increasing the amount of Tag (data not shown). The analytical results confirmed that the tag was polymerized, and attached to the polymer backbone. The tag addition mechanism was likely by chain transfer reaction between the growing polymer chain end and fluorescein. The fluorescence yield of fluorescein was very high, so a small tag dosing level could be used in polymer tagging.

### Example 3 Verification in Synthetic Bring and MQ (Milli-Q) Water

The fluorescence measurement confirmed very good sensitivity for the tagged polymers of Example 1 in MQ water.

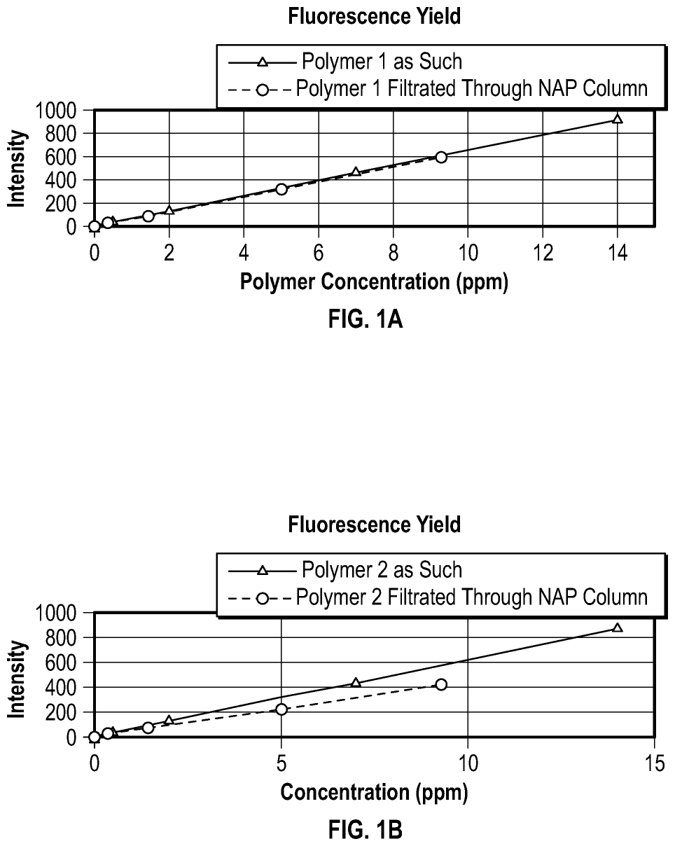

The attachment of the tag to the polymer backbone, as depicted at FIG. 1A and FIG. 1B, was estimated by comparing the fluorescence yield of clean and uncleaned samples. The samples were cleaned with a NAP™-25 size-exclusion chromatography (SEC) column (GE Healthcare, USA). The SEC column permitted polymeric materials to be separated from unattached tag monomers. As depicted at FIG. 1A and FIG. 1B, about 80-88% (by weight) of the fluorescein tag was incorporated into the polymer backbone.

### Example 4—Validation In Field Water

The validation of fluorescence detectability was carried out in produced water received from an oil field.

Fluorescence Measurement—Fluorescence measurements in this example were performed with the following parameters.

Instrument conditions were as follows:

$$λ ( excitation ) = 490 nm$$

$$λ ( emission range ) = 220 - 750 nm$$

$$λ ( fluorescein max . emission ) = 516 nm$$

$$Bandwidth = 5 nm$$

$$Sensitivity = high$$

$$Response time = 0.02 sec .$$

$$Speed = 5500 nm / min$$

$$Data interval = 2 nm$$

Sample preparation—No sample preparation was necessary; calibration solutions were measured as prepared. No NAP purification or pH adjustment was necessary.

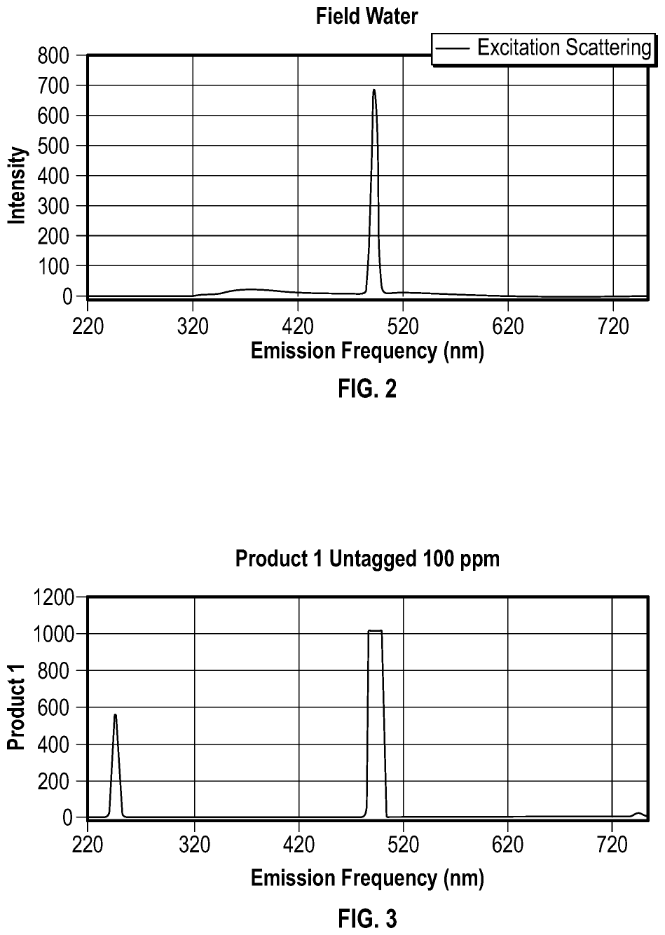

Field Water Fluorescence—To confirm the absence of background fluorescence, the filtered field water was measured and the results depicted in FIG. 2. No fluorescence at the nm peak emission of fluorescein was detected. Only a small amount of fluorescence at nm and the excitation scattering at 490 nm were detected.

Untagged Product 1 and Product 2—A 100 ppm solution of the untagged friction reducers was measured to confirm no fluorescence interference. No fluorescence was found in either sample. The scan of the Product 1 solution is depicted in FIG. 3.

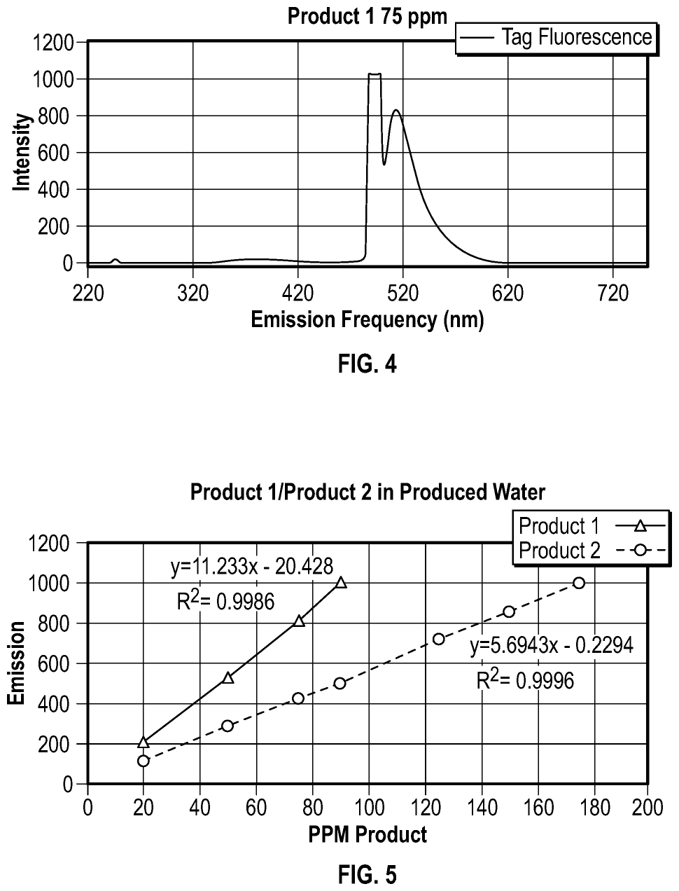

Calibration Samples—Measurements were made of the calibration standards up to the detector maximum intensity. An example of the scans is depicted in FIGS. 4 and 5. The intensity of the emission maximum was used to prepare a calibration curve of emission intensity vs. concentration. The emission intensity of the Product 1 was greater than the Product 2 at the same product concentration.

Both products exhibited good linearity over the measured ranges. The maximum range for detection of Product 1 was 90 ppm and for Product 2 was 175 ppm. Field samples containing higher concentrations could be diluted into the measurement range with water.

TABLE 3
Summary of intensities of two tagged polymers

| Polymer | | |
|---|---|---|
| concentration, ppm | Product 1 | Product 2 |
| 20 | 213 | 116 |
| 50 | 530 | 290 |
| 75 | 811 | 425 |
| 90 | 1004 | 500 |
| 125 | | 716 |
| 150 | | 852 |
| 175 | | 1000 |

Application data—The samples were tested in a flow loop using standard protocols for the EPAMs, which included measuring them in tap water and in a 30K synthetic brine at ppm.

Friction Reduction (FR) testing results on Product 1—_Two samples were tested in tap water and a 30,000 mg/L (30K) brine in the flow loop at 0.5gpt (500 ppm) using standard testing protocols. Both the tagged and the untagged samples had similar performance.

TABLE 4
Performance of tagged and untagged Product 1

| | | | | Time to | Time to |
|---|---|---|---|---|---|
| | FR dose | | Max FR | Max FR | 90% Max |
| FR Name | (gpt) | Brine | (%) | (s) | FR (s) |
| Product 1 - Untagged | 0.5 | Tap | 68 | 78 | 17 |
| Product 1 - Tagged | 0.5 | Tap | 69 | 70 | 17 |
| Product 1 - Untagged | 0.5 | 30K | 49 | 80 | 19 |
| Product 1 - Tagged | 0.5 | 30K | 51 | 42 | 17 |

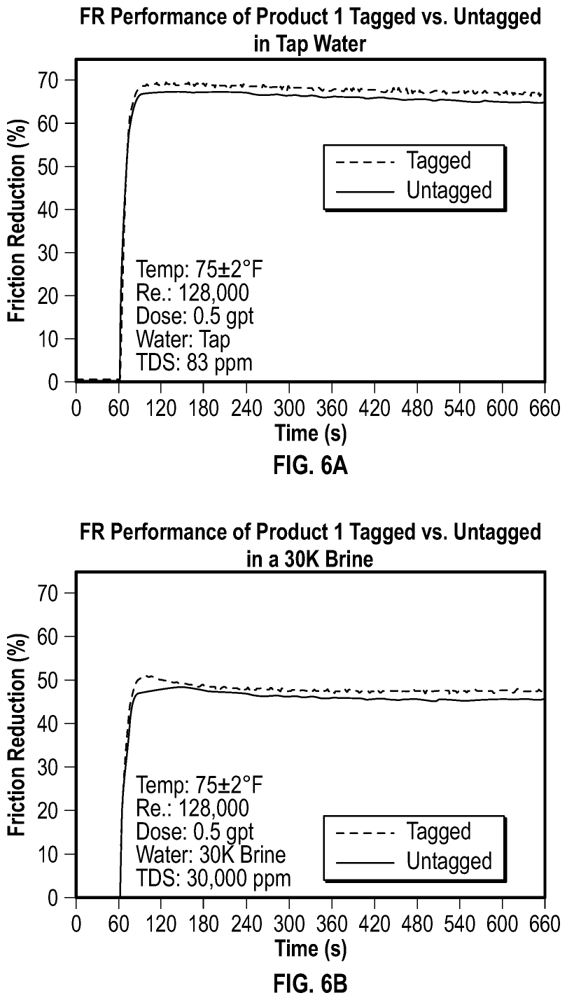

The performance of the tagged version of Product 1 was comparable to the untagged version, as depicted in FIG. 6A and FIG. 6B.

FR testing results on Product 2—The two samples were tested in tap water and a 30K brine in the flow loop at 0.5gpt (500 ppm) using standard testing protocols. Both the tagged and the untagged samples had similar performance.

TABLE 5
Performance of tagged and untagged Product 2

| | | | | Time to | Time to |
|---|---|---|---|---|---|
| | FR dose | | Max FR | Max FR | 90% Max |
| FR Name | (gpt) | Brine | (%) | (s) | FR (s) |
| Product 2 - Untagged | 0.5 | 30K | 68 | 30 | 13 |
| Product 2 - Tagged | 0.5 | 30K | 69 | 28 | 13 |
| Product 2 - Untagged | 0.5 | Tap | 71 | 42 | 15 |
| Product 2 - Tagged | 0.5 | Tap | 72 | 36 | 15 |

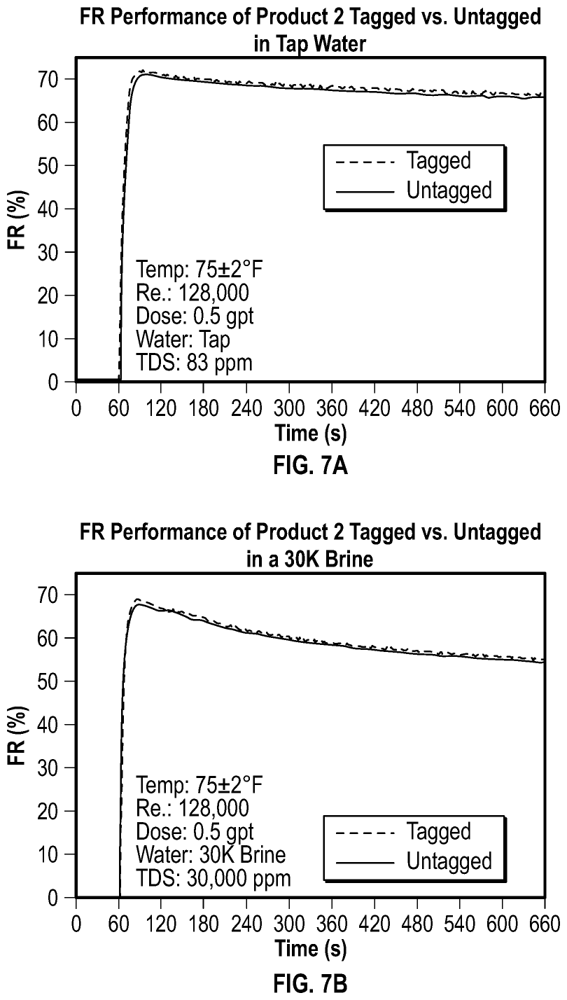

The performance of tagged and untagged Product 2 was comparable and the differences were minor, as depicted in FIG. 7A and FIG. 7B. Since there were both tagged and untagged samples in the same set, this comparison was helpful.

The tagged and the untagged friction reducers had similar performance in the flow loop when evaluated in tap water and in a 30K brine.

TABLE 6
Composition of 30K brine used for friction
reducer performance evaluation

| Ions in ppm | Tap | 30K Brine |
|---|---|---|
| Na⁺ | 13.91 | 11,356.27 |
| K⁺ | 3.00 | |
| Mg⁺⁺ | 2.00 | 96.94 |
| Ca⁺⁺ | 8.00 | 219.98 |
| Cl⁻ | 22.71 | 17,997.23 |
| HCO₃⁻ | 20.40 | 322.51 |
| SO₄⁻ | 13.00 | |
| Total hardness as CaCO₃ | 28.2 | 949.34 |
| TDS | 83.0 | 29,992.93 |

The following abbreviations are used herein: FR—Friction Reducer/Reduction; gpt —gallon per thousand gallon; Time to Max.FR—Time required to achieve the maximum friction reduction; and Time to 90% Max.FR—Time required to achieve 90% maximum friction reduction.

1. A method of hydraulic fracturing, the method comprising:
providing a well treatment fluid, wherein the well treatment fluid comprises a copolymer comprising—
(i) a first monomer comprising (a) a compound of formula (I), formula (I′), or formula (I″), or (b) a compound or an isomer of formula (II), wherein the first monomer is a tagging monomer, and
(ii) at least one second monomer comprising at least one polymerizable double bond or at least one polymerizable triple bond; 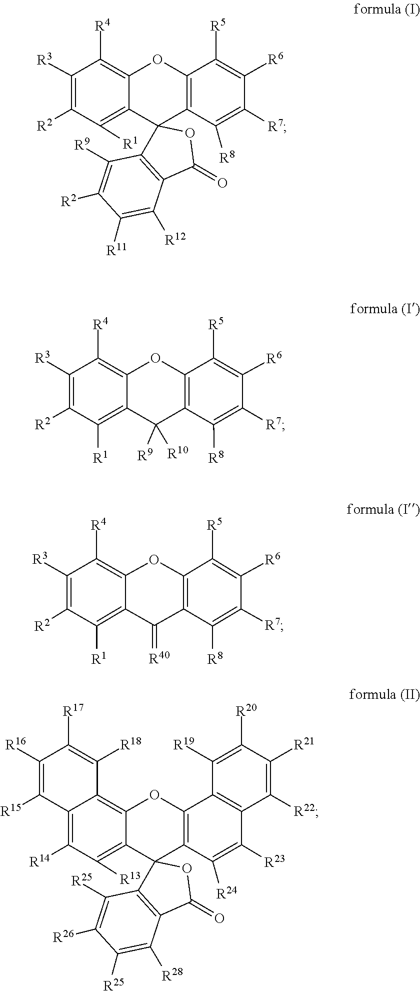
wherein R¹, R², R³, R⁴, R⁵, R⁶, R⁷, R⁸, R⁹, R¹⁰, R¹¹, R¹², R¹³, R⁴, R¹⁸, R¹⁶, R¹⁷, R¹⁸, R¹⁹, R²⁰, R²¹, R²², R²³, R²⁴, R²⁵, R²⁶, R²⁷, and R²⁸ are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl,
wherein R⁴⁰ is selected from the group consisting of C₁-C₆ alkoxy, C₂-C₆ alkenoxy, C₂-C₆ alkynoxy, —N(R′), —N(R′)(R″), C₂-C₆ alkyl, C₂-C₆ alkenyl, C₂-C₆ alkynyl, and C₄-C₁₄ aryl,
wherein R′ and R″ are independently selected from the group consisting of hydrogen and C₁-C₆ alkyl, and
wherein the isomers of Formula (II) comprise a compound of Formula (IIi), a compound of Formula (IIii), a compound of Formula (IIiii), or a combination thereof— 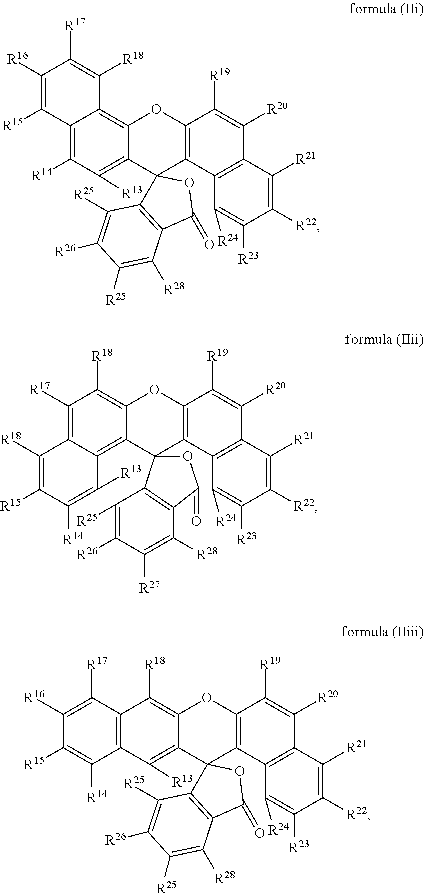
disposing the well treatment fluid in a well of a reservoir; and
measuring with an analytical technique an amount of the first monomer in the well treatment fluid after the disposing of the well treatment fluid in the well to (i) identify one or more producing stages, (ii) determine an optimum concentration of the copolymer in the well treatment fluid, or (iii) determine whether the well treatment fluid is recyclable.

2. The method of claim 1, wherein the analytical technique comprises fluorescence spectroscopy.

3. The method of claim 1, wherein the copolymer has a weight average molecular weight (M_{W}) of greater than 1,000,000 Da.

4. The method of claim 1, wherein the first monomer is present in the copolymer at an amount of about 0.005% to about 5%, by weight, based on the weight of the copolymer.

5. The method of claim 1, wherein the at least one second monomer comprises acrylamide, sodium acrylate, acrylic acid, sodium acrylamido-2-methylpropanesulfonic acid, or a combination thereof.

6. The method of claim 1, wherein the first monomer comprises the compound of formula (I), wherein R¹, R², R⁴, R⁵, R⁷, R⁸, R⁹, R¹⁰, R¹¹, and R¹² are hydrogen, or formula (I′) or (I″), wherein R¹, R², R⁴, R⁵, R⁷, and R⁸ are hydrogen.

7. The method of claim 6, wherein R³ and R⁶ are hydroxyl, or —N(R′)(R″).

8. The method of claim 1, wherein the copolymer is an anionic copolymer.

9. A method of hydraulic fracturing, the method comprising:
providing a first well treatment fluid;
providing a second well treatment fluid, wherein the first well treatment fluid and the second well treatment fluid comprise a copolymer selected independently from those comprising—
(i) a first monomer comprising (a) a compound of formula (I), formula (I′), or formula (I″), or (b) a compound or an isomer of formula (II), wherein the first monomer is a tagging monomer, and
(ii) at least one second monomer comprising at least one polymerizable double bond or at least one polymerizable triple bond; 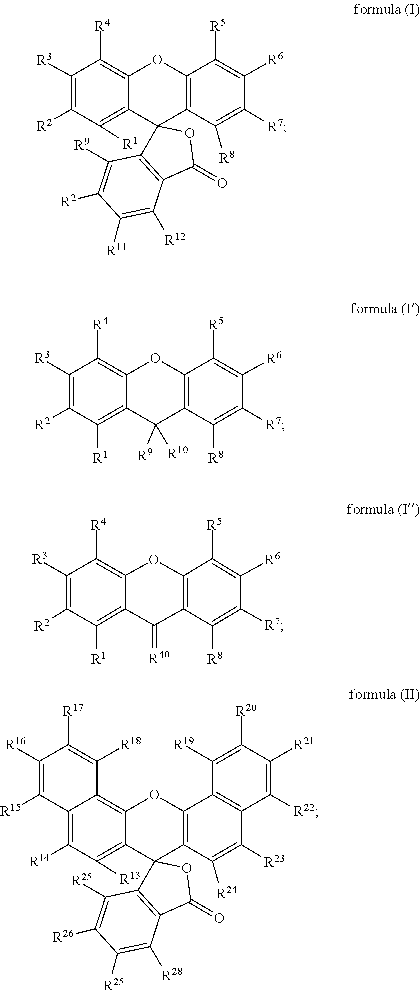
wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl,
wherein R⁴⁰ is selected from the group consisting of C₁-C₆ alkoxy, C₂-C₆ alkenoxy, C₂-C₆ alkynoxy, —N(R′), —N(R′)(R″), C₂-C₆ alkyl, C₂-C₆ alkenyl, C₂-C₆ alkynyl, and C₄-C₁₄ aryl,
wherein R′ and R″ are independently selected from the group consisting of hydrogen and C₁-C₆ alkyl, and
wherein the isomers of Formula (II) comprise a compound of Formula (IIi), a compound of Formula (IIii), a compound of Formula (IIiii), or a combination thereof— 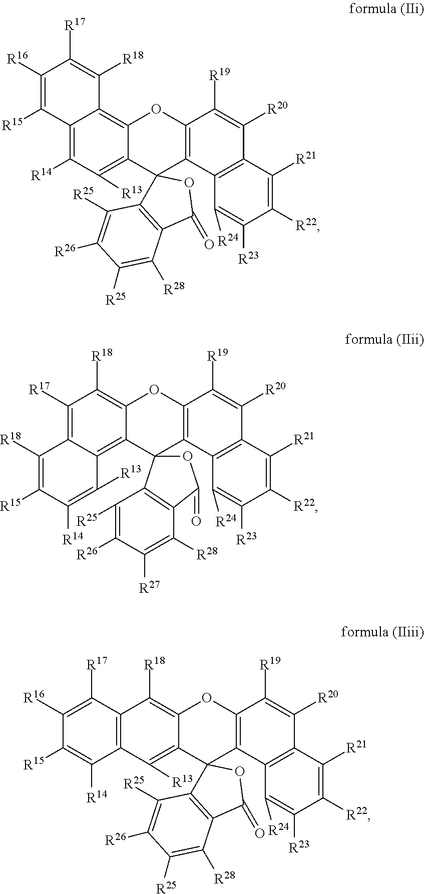
wherein (A) a peak fluorescence emission of the first monomer of the first well treatment fluid and (B) a peak fluorescence emission of the first monomer of the second well treatment fluid occur at different wavelengths;
disposing the first well treatment fluid in a first well of a reservoir;
disposing the second well treatment fluid in a second well of the reservoir; and
measuring with an analytical technique (i) an amount of the first monomer of the first well treatment fluid and (ii) an amount of the first monomer of the second well treatment fluid after the disposing of the first and the second well treatment fluids in the first and the second wells, respectively, to determine (a) a degree of connectivity between the first well and the second well of the reservoir, or (b) one or more producing stages of the first well or the second well.

10. The method of claim 9, wherein the measuring with the analytical technique occurs during flow back operations.

11. The method of claim 9, wherein the analytical technique comprises fluorescence spectroscopy.

12. The method of any one of claims 1 to 11, wherein the copolymer is present in the well treatment fluid, the first well treatment fluid, or the second well treatment fluid at an amount of about 5 ppm to about 1,000 ppm.

13. A method of polymerization, the method comprising:
providing an aqueous phase having a first pH, the aqueous phase comprising water and at least one second monomer;
optionally adjusting the first pH to a second pH;
disposing a first monomer in the aqueous phase;
providing an oil phase comprising an oil and at least one emulsifying surfactant;
combining the aqueous phase and the oil phase to form an emulsion;
optionally deoxygenizing the emulsion;
disposing an initiator in the emulsion; and
contacting the emulsion with an initiator to copolymerize the first monomer and the at least one second monomer to form the copolymer in the emulsion;
wherein (i) the first monomer comprises (a) a compound of formula (I), formula (I′), or formula (I″), or (b) a compound or an isomer of formula (II), and
(ii) the at least one second monomer comprises at least one polymerizable double bond or at least one polymerizable triple bond; 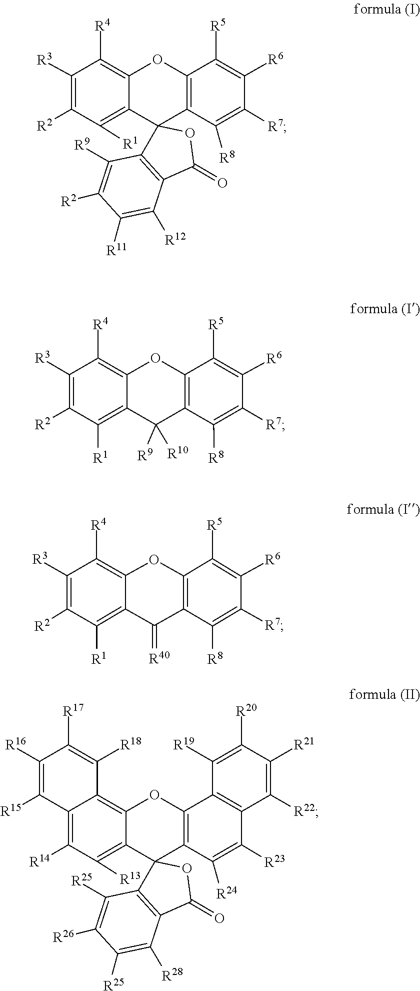
wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently selected from the group consisting of hydrogen, hydroxyl, C₁-C₆ alkoxy, C₂-C₆ alkenoxy, C₂-C₆ alkynoxy, —N(R′)(R″), C₁-C₆ alkyl, C₂-C₆ alkenyl, C₂-C₆ alkynyl, and C₄-C₁₄ aryl,
wherein R⁴⁰ is selected from the group consisting of C₁-C₆ alkoxy, C₂-C₆ alkenoxy, C₂-C₆ alkynoxy, —N(R′), —N(R′)(R″), C₂-C₆ alkyl, C₂-C₆ alkenyl, C₂-C₆ alkynyl, and C₄-C₁₄ aryl,
wherein R′ and R″ are independently selected from the group consisting of hydrogen and C₁-C₆ alkyl, and
wherein the isomers of Formula (II) comprise a compound of Formula (IIi), a compound of Formula (IIii), a compound of Formula (IIiii), or a combination thereof—

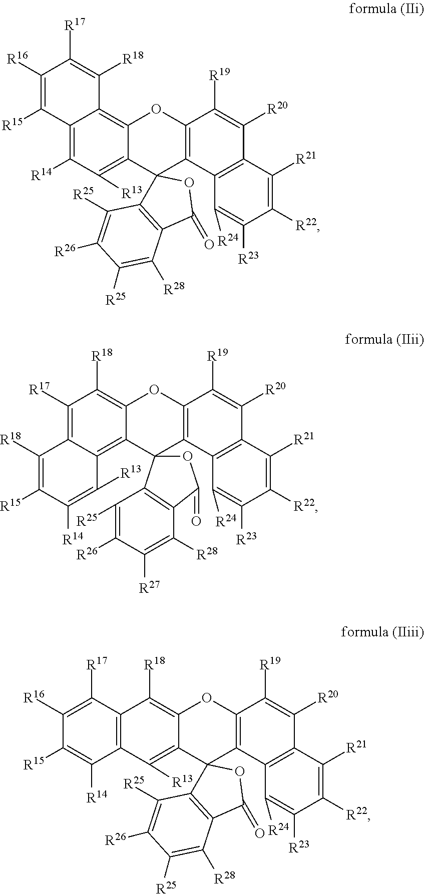

14. The method of claim 13, further comprising removing unreacted monomers from the emulsion.

15. The method of claim 13, further comprising disposing in the emulsion an inverting surfactant package.
